(19) United States
(12) Patent Application Publication
(10) Pub. No.: US 2024/0141326 A1
(43) Pub. Date: May 2, 2024

(54) MULTIPLEXING OF EXPERIMENTAL CONDITIONS AND SAMPLES IN SPATIAL GENOMICS

(71) Applicant: CALIFORNIA INSTITUTE OF TECHNOLOGY

(72) Inventors: Daniel DAR, Pasadena, CA (US); Dianne K. NEWMAN, Pasadena, CA (US); Kirsten FRIEDA, Pasadena, CA (US); Long CAI, Pasadena, CA (US)

(21) Appl. No.: 18/278,586

(22) PCT Filed: Feb. 24, 2022
(86) PCT No.: PCT/US2022/017757

§ 371 Date: Aug. 23, 2023

Related U.S. Application Data
(60) Provisional application No. 63/153,234, filed on Feb. 24, 2021.

(51) Int. Cl. C12N 15/10

(52) U.S. Cl. CPC C12N 15/1065; C12N 15/1055; C12N 15/1058

(57) ABSTRACT

Method for detecting and barcoding the molecular changes occurring in two or more samples upon exposure to different stimuli. This disclosure herein sets for methods that allow a targeted transcriptome-imaging approach that records both gene-expression and spatial context within microscale assemblies at a single-cell and molecule resolution on biological samples. This disclosure herein sets for methods that allows the application to a variety of biological samples for the study of cellular processes, growth, and interactions between biological samples.

### CROSS-REFERENCE TO RELATED APPLICATIONS

This application claims the benefit of U.S. Provisional Patent Application No. 63/153,234, filed Feb. 24, 2021. The contents of the above-referenced application are hereby incorporated by reference in their entirety.

### STATEMENT REGARDING FEDERALLY-SPONSORED RESEARCH

This invention was made with government support under Grant No(s). AI127850 & HL152190 awarded by the National Institutes of Health and under Grant No. W911NF-17-1-0024 awarded by the US Army. The government has certain rights in the invention.

### FIELD OF INVENTION

The present disclosure provides methods for detecting and barcoding the molecular changes occurring in two or more samples upon exposure to different stimuli.

### BACKGROUND

Recent advances in imaging methods provide a means to chart the physical associations between different species in natural environments (Mark Welch et al., 2016; Shi et al., 2020; Tropini et al., 2017; Wilbert et al., 2020). However, interpreting these maps remains challenging without additional functional information on the physiological states and activities of relevant community members. In contrast, recent adaptions of eukaryotic single-cell RNA-sequencing (scRNA-seq) approaches provide a powerful means of exploring the phenotypic landscape of planktonic bacteria (Blattman et al., 2020; Imdahl et al., 2020; Kuchina et al., 2021). However, these approaches do not preserve the spatial context of analyzed cells and are therefore limited in their capacity to address single and multispecies biofilms. Thus, a major gap exists in our ability to account for both spatial and functional complexity, limiting progression toward a high-resolution understanding of microbial life.

Single-molecule fluorescence in situ hybridization (FISH) based technologies have been used to measure gene-expression directly within native tissues, recording both spatial and functional information. However, while these methods have shed important light on single-cell heterogeneity they have been traditionally limited to measuring the expression of only a few genes at a time (Choi et al., 2014; Femino et al., 1998; Raj et al., 2008; So et al., 2011). In addition to this limited throughput, single-gene measurements do not provide a means to capture coordinated cellular responses—the molecular “fingerprint” of multiple biological activities that underpin distinct physiological states. Recent advances in combinatorial mRNA labeling and sequential FISH (seqFISH) now allow for hundreds and even thousands of genes to be analyzed within the same sample at a sub-micron resolution (Chen et al., 2015; Eng et al., 2019; Lubeck et al., 2014).

There is a need for methods that can detect and analyze the enormous molecular changes that can occur between different biological samples when exposed to different conditions.

### SUMMARY

The present disclosure provides methods for detecting and barcoding the molecular changes occurring in two or more samples upon exposure to different stimuli. This disclosure sets forth processes and using the same, and other solutions to problems in the field.

In some embodiments, a method is disclosed herein providing two or more samples of cells. In some embodiments, the method comprises labelling the cells of each sample with one or more sample probes, wherein the sample probes interact with one or more sample identifiers. In some embodiments, the method comprises treating each sample in the two or more samples to different conditions. In some embodiments, the method comprises combining the two or more samples to create a pooled sample. In some embodiments, the method comprises barcoding one or more targets in the pooled sample. In some embodiments, the method comprises imaging the barcodes. In some embodiments, the method comprises demultiplexing the sample probes to associate cells with their samples.

In some embodiments, the method of any of the previous embodiments comprises labelling the cells of each sample with one or more sample probes, wherein the sample probes interact with one or more sample identifiers after treating each sample in the two or more samples to different conditions.

In some embodiments, the method of any of the previous embodiments comprises barcoding one or more targets in the samples before combining the two or more samples to create a pooled sample.

In some embodiments, the method of any of the previous embodiments comprises demultiplexing the sample probes to associate cells with their samples before imaging the barcodes.

In some embodiments, the methods of any of the previous embodiments comprises demultiplexing the sample probes to associate cells with their samples before barcoding one or more targets in the pooled sample.

The embodiments of the methods described herein allow a targeted transcriptome-imaging approach that records both gene-expression and spatial context within microscale assemblies at a single-cell and molecule resolution on biological samples. In certain embodiments, the methods described herewithin allow the application of this to a variety of biological samples for the study of cellular processes, growth, and interactions between biological samples. In certain embodiments, the methods disclosed herewithin allow the identification and analysis of metabolic and virulence related cell-states that emerge dynamically during growth. In certain embodiments, the methods disclosed herein allow processes including motility and kin-exclusion mechanisms and identify extensive and highly spatially-resolved metabolic heterogeneity to be studied.

### DETAILED DESCRIPTION

The following description is presented to enable one of ordinary skill to make and use the disclosed subject matter and to incorporate it in the context of applications. Various modifications, as well as a variety of uses in different applications, will be readily apparent to those skilled in the art, and the general principles defined herein may be applied to a wide range of embodiments. Thus, the present disclosure is not intended to be limited to the embodiments presented, but is to be accorded the widest scope consistent with the principles and novel features disclosed herein.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1: Parallel and sequential mRNA-FISH in bacteria. (A) seqFISH probe design scheme. Primary probes contain unique sequences (Si) that are read by secondary probes (colored wands). Each gene is read by a unique probe and its fluorescence can be turned “ON” or “OFF”. (B) mRNA-FISH applied sequentially to the same sample. In each cycle, a new set of secondary readout probes are introduced. Raw fluorescence data is shown on the right and the detected local spot maxima are shown in the spot detection image. Merged spots for many genes shown in shuffled colors. (C) Combinatorial labeling can be used to encode species taxonomy using 16S rRNA, or to enable the parallel study of (D) bacteria grown in different conditions.

FIG. 2: Parallel seqFISH (par-seqFISH) of an LB growth curve experiment (A) The sampled LB growth curve. Collected time points are indicated with gray circles. A zoom-in shows the sampled lag phase. (B) Demultiplexed bacteria and their mRNAs. The merged, raw Ribo-Tag 16S rRNA fluorescence is shown for a representative region. Different barcodes (16S combinations) result in unique colors that visually report the condition of which they originated from (indicated with the corresponding OD600 value). Ellipses fitted to the segmented cell boundaries are shown. The mRNA spots (fitted position of maximal intensity) for all genes per cell are shown in unique colors per gene. Each spot may represent more than one mRNA copy. (C-F) Condition specific distributions of nucleoid length, chromosome copy, ribosome levels and total mRNAs detected across our gene set. (G) Heatmap showing average gene expression normalized to the maximal value for each gene across all conditions. Highlighted gene groups and their functions are indicated on the right.

FIG. 3: Single-bacterium analysis reveals physiologically distinct dynamic sub-populations. (A) UMAP analysis using cells from all 11 time points. Identified clusters are shown in different colors and are indexed by group size. Specific group functions and numbers are shown. (B) Gene expression overlays for four genes that report on metabolic state, stationary phase progression and exoproduct biosynthesis. (C-F) Density scatter plots of cells from individual conditions in a zoom-in of the UMAP (dashed box in panel A). The clusters are indicated by their index. (G-J) Gene expression overlays shown as B and indicated in the figure.

FIG. 4: Spatial transcriptomics in P. aeruginosa biofilms at a single cell resolution. (A) A representative field of view collected during a 10 h surface colonization experiment showing cells via 16SrRNA fluorescence (gray). A zoom-in (orange box) shows the cell segmentation masks depicted as white ellipses. The 16S-rRNA signal and mRNA-FISH data for several genes are shown in different colors. (B) A 35 h experiment field is shown in an identical manner to panel A. Scale bar length is annotated within the figure. (C) Joint UMAP cluster analysis of biofilm and planktonic experiments. Planktonic cells are shown for all time points collected (D) UMAP scatter plots showing cells from either planktonic or biofilm experiments as indicated. Below, a highlighted set of UMAP clusters associated with each experiment is annotated. (E-H) UMAP overlay with specific gene data.

FIG. 5: Spatial expression patterns for motility and pyocin related genes (A-B) Representative regions from the 10 h and 35 h biofilm experiments, cells are shown in via 16S-rRNA fluorescence (gray) and overlayed with raw mRNA-FISH fluorescence for different genes as indicated. (C) planktonic cells from the pair liquid experiments. Cells are shown via DAPI and expression as indicated (D-E) 10 h aggregate showing R2-pyocin expression. (F) Enrichment of R2-Pyocin mRNA near strong induction sites (cell with 99.5th percentile pyocin expression). X-axis shows the number of cells closest to an induction site that were analyzed (neighborhood size; center cell was excluded). Y-axis shows the enrichment in each neighborhood relative to the total population. A non-pyocin control gene is shown (rpoA). (G) Examples of mRNA R-pyocin transcript and ribosome polar localization as indicated in the legends.

FIG. 6: Oxygen availability shapes microscale metabolic heterogeneity in biofilms (A-E) Representative 10 h biofilms. Cells are shown via 16S-rRNA fluorescence (gray) and overlayed with raw mRNA-FISH fluorescence for different genes as indicated in each panel. White circles highlight regions of interest. (F) Cells painted according to their UMAP derived metabolic state as indicated in the panel legends (also see FIG. 14 clusters, 0, 8, 12 and 15), showing co-localization of multiple metabolic states within a given region.

FIG. 7: Functional zonation in a single micro-aggregate. (A-D) A P. aeruginosa 35 h aggregate. Bacteria are shown via 16S-rRNA fluorescence (gray) and are overlaid with raw mRNA-FISH fluorescence for different genes as described in the panel legends.

FIG. 8: Multiplexing mammalian cell samples using probes that label ribosomal RNA. Here, three suspended samples of mouse embryonic stem cells (mESCs) where separately hybridized with a unique probe against 18S rRNA. The combined samples were then distinguished by imaging their different 18S rRNA probes, shown as red, dark blue, or no additional color beyond the DAPI stain highlighting all nuclei (cyan).

FIG. 9: (A) Examples of positive signal for genes labeled with one of the three fluorophores used in this study A647 (red), cy3B (green), and A488 (cyan). For context, the mRNA-FISH fluorescence is shown over DAPI (dark silhouette). (B) Same regions as is in panel A but showing the raw fluorescence of the negative control genes for each fluorophore. For direct comparison, the intensity range is identical between positive and negative panels in A-B. Scale bar represents 5 μm.

FIG. 10: Single-cell dispersions in UMAP space for each growth curve time point. A UMAP density plot of cells belonging to specific time points. The OD600 values and the number of the time points are shown over each plot. Color intensity represents cell density.

FIG. 11: Distributions of single-cell parameters across the detected UMAP clusters Distributions of nucleoid length, chromosome copy, ribosome levels and total mRNAs for each of the UMAP clusters described in main FIG. 3.

FIG. 12: Spatial correlation analysis. Gene centered neighborhood analysis for detecting spatial correlation. For each gene, its 99th percentile expressing cells were identified and their 5 immediate neighbors within 3 μm were collected (leaving out the enriched center cell). The set of all such neighbors cross the experiment was analyzed together to produce a mean expression profile that was compared with the total population to produce a local enrichment/depletion ratio. The Pearson correlation between such gene neighborhood profiles was calculated and shown above as a clustered heat map. Five selected regions are highlighted and numbered. Key genes within each cluster are described to the right.

FIG. 13: Distributions of single-cell parameters per UMAP cluster (A-B) Representative 10 h microaggregates. Cells are shown via 16S rRNA FISH fluorescence (gray) and overlayed with gene-expression as indicated in each panel. White circles highlight regions of interest. (C-D) Zoom-in on region 1 and 2 showing uspL (cyan) and sigX (red). (E) Cells painted according to their neighborhood class as indicated in the panel legend. (F-H) Zoom-in highlighted regions overlaid with raw gene-expression as indicated in the panel legends.

FIG. 14: UMAP analysis of 10 h biofilms (A) UMAP analysis was performed using the single neighborhood profiles. Below, clusters are labeled and are divided into predicted anaerobic groups. (B) UMAP overlaid with specific gene data. The cluster number positions are shown in the figure.

FIG. 15: Functional zonation in 35 h microaggregates (A-B) Various P. aeruginosa 35 h aggregate. Bacteria are shown via 16S rRNA FISH fluorescence (gray) and are overlaid with raw mRNA-FISH fluorescence for several genes as described in the images.

### DEFINITIONS

Unless otherwise noted, terms are to be understood according to conventional usage by those of ordinary skill in the relevant art.

As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

The term “oligonucleotide” refers to a polymer or oligomer of nucleotide monomers, containing any combination of nucleobases, modified nucleobases, sugars, modified sugars, phosphate bridges, or modified bridges. Oligonucleotides can be of various lengths. In particular embodiments, oligonucleotides can range from about 2 to about 1000 nucleotides in length. In various related embodiments, oligonucleotides, single-stranded, double-stranded, and triple-stranded, can range in length from about 4 to about 10 nucleotides, from about 10 to about 50 nucleotides, from about 20 to about 50 nucleotides, from about 15 to about 30 nucleotides, from about 20 to about 30 nucleotides in length. In some embodiments, the oligonucleotide is from about 9 to about 39 nucleotides in length. In some embodiments, the oligonucleotide is at least 4 nucleotides in length. In some embodiments, the oligonucleotide is at least 5 nucleotides in length. In some embodiments, the oligonucleotide is at least 6 nucleotides in length. In some embodiments, the oligonucleotide is at least 7 nucleotides in length. In some embodiments, the oligonucleotide is at least 8 nucleotides in length. In some embodiments, the oligonucleotide is at least 9 nucleotides in length. In some embodiments, the oligonucleotide is at least 10 nucleotides in length. In some embodiments, the oligonucleotide is at least 11 nucleotides in length. In some embodiments, the oligonucleotide is at least 12 nucleotides in length. In some embodiments, the oligonucleotide is at least 15 nucleotides in length. In some embodiments, the oligonucleotide is at least 20 nucleotides in length. In some embodiments, the oligonucleotide is at least 25 nucleotides in length. In some embodiments, the oligonucleotide is at least 30 nucleotides in length. In some embodiments, the oligonucleotide is a duplex of complementary strands of at least 18 nucleotides in length. In some embodiments, the oligonucleotide is a duplex of complementary strands of at least 21 nucleotides in length.

As used herein, the term “probe” or “probes” refers to any molecules, synthetic or naturally occurring, that can attach themselves directly or indirectly to a molecular target (e.g., an mRNA sample, DNA molecules, protein molecules, RNA and DNA isoform molecules, single nucleotide polymorphism molecules, and etc.). For example, a probe can include a nucleic acid molecule, an oligonucleotide, a protein (e.g., an antibody or an antigen binding sequence), or combinations thereof. For example, a protein probe may be connected with one or more nucleic acid molecules to form a probe that is a chimera. As disclosed herein, in some embodiments, a probe itself can produce a detectable signal. In some embodiments, a probe is connected, directly or indirectly via an intermediate molecule, with a signal moiety (e.g., a dye or fluorophore) that can produce a detectable signal. In some embodiments, a “probe” may be a small molecule.

As used herein, the term “binding sites” refer to a portion of a probe where other molecules may bind to the probe. In certain embodiments, the binding sites of a probe bind to another molecule through a non-covalent interaction.

As used herein, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample is or comprises bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. In some embodiments, the term “sample” refers to a nucleic acid such as DNA, RNA, transcripts, or chromosomes. In some embodiments, the term “sample” refers to nucleic acid that has been extracted from the cell.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

As disclosed herein, the term “label” generally refers to a molecule that can recognize and bind to specific target sites within a molecular target in a cell. For example, a label can comprise an oligonucleotide that can bind to a molecular target in a cell. The oligonucleotide can be linked to a moiety that has affinity for the molecular target. The oligonucleotide can be linked to a first moiety that is capable of covalently linking to the molecular target. In certain embodiments, the molecular target comprises a second moiety capable of forming the covalent linkage with the label. In particular embodiments, a label comprises a nucleic acid sequence that is capable of providing identification of the cell which comprises or comprised the molecular target. In certain embodiments, a plurality of cells is labelled, wherein each cell of the plurality has a unique label relative to the other labelled cells. In some embodiments, the term “moeity” refers to the term “label.”

As used herein, the term “dot” refers to the single molecules that fluoresce as they are excited by light of a particular frequency. In certain embodiments, a nucleic acid or protein, or combination thereof hybridized to a oligonucleotide probe or protein probe or combination thereof produces a excitation “dot” when excited by a particular frequency.

As disclosed herein, the term “bridge” refers to probes that are complementary to other probes that may hybridize or bind to the complementary probes, the other probes are not covalently linked 5′ to 3′ to each other. In certain embodiments, the term “bridge probe” uses the definition and techniques of Lohman et al. Efficient DNA ligation in DNA-RNA hybrid helices by Chlorella virus DNA ligase. Nucleic Acid Research, 2014, vol. 42 No. 3 1831-1844, incorporated by its entirety.

As used herein, the term “antibody” refers to any macromolecule that would be recognized as an antibody by those of skill in the art. In some embodiments, an antibody includes any form of an antibody other than the full length form that would be recognized by an antibody fragment by those of skill in the art.

As used herein, the term “bind” refers to two or more proteins, oligonucleotides, small molecules, antibodies, and combinations thereof to interact to form larger complexes. In certain embodiments “bind” refers to the hybridization of oligonucleotides with RNA, DNA, oligonucleotides, or combinations thereof.

As used herein, the term “growth conditions” refers to the factors influencing the proliferation or viability of cells in a population. In some embodiments, the factors include, but are not limited to temperature, aeration, pressure, light, and nutrients.

As used herein, the term “chemical exposures” refers to any chemical or chemicals added to a population of cells that influences the proliferation or viability of cells in a population. In some embodiments, some embodiments, the factors include, but are not limited to drugs or pharmaceuticals. In certain embodiments, the drugs or pharmaceuticals are those listed on the United States Food and Drug Administration's database.

As used herein, the term “environmental conditions” refers to the factors derived from the environment influencing influences the proliferation or viability of cells in a population. In some embodiments, the conditions include, but are not limited to including the soil, groundwater, surface water or ambient air, or hazardous material, wherein the hazardous material exceeds a level of any applicable standard or threshold under any Environmental Law.

As used herein, the term “sample identifier” refers to a cellular component that can be used to identify a specific biological sample when combined with different biological samples. In some embodiments, the sample identifiers are selected from synthetic RNA, ribosomal RNA, 16S RNA, 18S RNA, and lncRNA, or any combination thereof. In some embodiments, the sample identifier comprises 16SRNA.

As used herein, the term “sample probes” refers to proteins, modified proteins, RNA, oligo-nucleotides, antibodies, antibody fragments, or combinations thereof that interact with a sample identifier to identify a specific biological sample, wherein the biological sample has been pooled with different biological samples. In some embodiments, the sample probe interacts with the sample identifier by hybridization.

As used herein, the term “demultiplexing” refers to a process where sample probes interacting with sample identifiers and their associated cells are associated with different targets and their corresponding barcodes.

### Overview

The disclosure herein provides methods for detecting and barcoding the molecular changes occurring in two or more samples upon exposure to different stimuli. This disclosure herein sets forth embodiments to multiplex thousands of genomic and cellular features in molecular samples by using a molecular barcode that can be readout by sequential rounds of imaging to identify the sample barcodes.

In some embodiments, a method is disclosed herein providing two or more samples of cells. In some embodiments, the method comprises labelling the cells of each sample with one or more sample probes, wherein the sample probes interact with one or more sample identifiers. In some embodiments, the method comprises treating each sample in the two or more samples to different conditions. In some embodiments, the method comprises combining the two or more samples to create a pooled sample. In some embodiments, the method comprises barcoding one or more targets in the pooled sample. In some embodiments, the method comprises imaging the barcodes. In some embodiments, the method comprises demultiplexing the sample probes to associate cells with their samples.

In some embodiments, the method of any of the previous embodiments comprises labelling the cells of each sample with one or more sample probes, wherein the sample probes interact with one or more sample identifiers after treating each sample in the two or more samples to different conditions.

In some embodiments, the method of any of the previous embodiments comprises barcoding one or more targets before combining the two or more samples to create a pooled sample.

In some embodiments, the method of any of the previous embodiments comprises demultiplexing the sample probes to associate cells with their samples before imaging the barcodes.

In some embodiments, the methods of any of the previous embodiments comprises demultiplexing the sample probes to associate cells with their samples before barcoding one or more targets in the pooled sample.

### Samples

In some embodiments, the methods of any of the embodiments described herein are capable of analyzing multiple samples at the same time.

In certain embodiments, the method comprises analyzing two or more samples. In certain embodiments, the method comprises analyzing at least 3, 4, 5, 6, 7, 8, 9, 10, 11, 12, 13, 14, 15, 20, or 25 different samples. In certain embodiments, the method comprises combining the two or more samples to create a pooled sample.

In some embodiments, the method comprises analyzing samples, wherein the samples comprise bacterial cells, archaeal cells, eukaryotic cells, or a combination thereof. In certain embodiments, the samples comprise tissues, cells, or extracts from cells. In certain embodiments, the samples comprise biofilms. In certain embodiments, the samples comprise cells obtained from patients.

In some embodiments, the method comprises engineering the samples to comprise exogenous sample identifiers that can be barcoded for identification. In some embodiments, the samples comprise endogenous sample identifiers that can be barcoded for identification. In some embodiments, the samples comprise exogenous sample identifiers and endogenous sample identifiers that can be barcoded for identification.

In some embodiments, the method comprises engineering the samples so that specific genes can be turned “ON” or turned “OFF” in the presence of a signal. In certain embodiments, genes that are turned “ON” express more RNA than genes that are turned “OFF.” In certain embodiments, genes that are turned “OFF” express less RNA than genes that are turned “OFF.”

In some embodiments, the method comprises treating each sample to different growth conditions, different chemical exposures, different environmental conditions, or combinations thereof. In certain embodiments, the different growth conditions include, but are not limited to temperature, aeration, pressure, light, and nutrients. In certain embodiments, different chemical exposures include, but are not limited to drugs or pharmaceuticals. In certain embodiments the drugs or pharmaceutical may include, but are not limited to antibiotics, cancer therapeutics, anti-fungals, hormones, and vitamins. In certain embodiments, different environmental conditions include, but are not limited to the soil, groundwater, surface water or ambient air, or hazardous material, wherein the hazardous material exceeds a level of any applicable standard or threshold under any Environmental Law.

### Sample Identifier

In some embodiments, the method comprises a sample identifier to identify the sample of interest. In certain embodiments, the sample identifiers differentiate one organism from another. In certain embodiments, the sample identifier differentiates one sample from another. In certain embodiments, the sample identifier differentiates one sample from another, wherein each sample has been subjected to different stimuli. In certain embodiments, the sample identifier is introduced into an organism through molecular biology techniques.

In some embodiments, the method comprises using sample identifiers that are selected from transcripts, RNA, DNA loci, chromosomes, DNA, proteins, lipids, glycans, cellular targets, organelles, exogenous elements, and any combinations thereof. In certain embodiments, exogenous sample identifiers are selected from transcripts, RNA, DNA loci, chromosomes, DNA, proteins, lipids, glycans, cellular targets, organelles, exogenous elements, and any combinations thereof.

In some embodiments, the sample identifier comprises, synthetic RNA, ribosomal RNA, 16S RNA, 18S RNA, and lncRNA. In some embodiments, the sample identifier comprises 16SRNA. In some embodiments, the sample identifier comprises 18S RNA. In some embodiments, the sample identifier is synthetic RNA. In some embodiments, the sample identifier is ribosomal RNA. In some embodiments, the sample identifier is lncRNA.

In some embodiments, the sample identifier interacts with one or more sample intermediate probes.

In some embodiments, the sample identifier comprises a sample readout probe binding site or an intermediate probe binding site.

### Sample Probes

In some embodiments, the method comprises using a sample probe, wherein the sample probe interacts with the sample identifier in order to identify the sample of interest.

In some embodiments, each sample probe is selected from proteins, modified proteins, RNA, oligonucleotides, antibodies, antibody fragments, and combinations thereof.

In some embodiments, each sample probe comprises an oligonucleotide. In some embodiments the oligonucleotides comprise sequences that interact with identifier regions on sample identifiers by hybridization. In some embodiments, the oligonucleotide comprises a sequence that is complementary to the sample identifier region, and wherein the sequence complementarity is at least 60%, 65%, 70%, 75%, 80%, 85%, 90%, 91%, 92%, 93%, 94%, 95%, 96%, 97%, 98%, 99%, or 100%.

In some embodiments, the sample probe comprises oligonucleotides that are at least 5 nucleotides long. In some embodiments, the sample probe comprises oligonucleotides that are at least 6 nucleotides long. In some embodiments, the sample probe comprises oligonucleotides that are at least 7 nucleotides long. In some embodiments, the sample probe comprises oligonucleotides that are at least 8 nucleotides long. In some embodiments, the sample probe comprises oligonucleotides that are at least 9 nucleotides long. In some embodiments, the sample probe comprises oligonucleotides that are at least 10 nucleotides long. In some embodiments, the sample probe comprises oligonucleotides that are at least 11 nucleotides long. In some embodiments, the sample probe comprises oligonucleotides that are at least 12 nucleotides long. In some embodiments, the sample probe comprises oligonucleotides that are at least 13 nucleotides long. In some embodiments, the sample probe comprises oligonucleotides that are at least 14 nucleotides long. In some embodiments, the sample probe comprises oligonucleotides that are at least 15 nucleotides long. In some embodiments, the sample probe comprises oligonucleotides that are at least 16 nucleotides long. In some embodiments, the sample probe comprises oligonucleotides that are at least 17 nucleotides long. In some embodiments, the sample probe comprises oligonucleotides that are at least 18 nucleotides long. In some embodiments, the sample probe comprises oligonucleotides that are at least 19 nucleotides long. In some embodiments, the sample probe comprises oligonucleotides that are at least 20 nucleotides long. In some embodiments, the sample probe comprises oligonucleotides that are at least 21 nucleotides long. In some embodiments, the sample probe comprises oligonucleotides that are at least 22 nucleotides long. In some embodiments, the sample probe comprises oligonucleotides that are at least 23 nucleotides long. In some embodiments, the sample probe comprises oligonucleotides that are at least 24 nucleotides long. In some embodiments, the sample probe comprises oligonucleotides that are at least 25 nucleotides long. In some embodiments, the sample probe comprises oligonucleotides that are at least 26 nucleotides long. In some embodiments, the sample probe comprises oligonucleotides that are at least 27 nucleotides long. In some embodiments, the sample probe comprises oligonucleotides that are at least 28 nucleotides long. In some embodiments, the sample probe comprises oligonucleotides that are at least 29 nucleotides long. In some embodiments, the sample probe comprises oligonucleotides that are at least 30 nucleotides long. In some embodiments, the sample probes of any of the previous embodiments comprises oligonucleotides that are less than 35, 40, 45, 50, 100 nucleotides in length.

In some embodiments, the sample probe comprises a detectable label. In some embodiments, the sample probe hybridizes to the sample identifier. In some embodiments, the sample probe is sample readout probe. In some embodiments, the sample probe hybridizes to an intermediate sample probe.

### Readout Probes

In some embodiments, the method comprises using a sample readout probe that interacts with the sample probe in order to differentiate the samples. In some embodiments, the method comprises using a target readout probe to barcode a target.

In some embodiments, the readout probe is selected from proteins, modified proteins, RNA, oligonucleotides, antibodies, antibody fragments, and combinations thereof. In some embodiments, the readout probe further comprises a detectably moiety.

In some embodiments, the readout probe comprises an oligonucleotide with a detectably moiety.

In some embodiments, the readout probe comprises oligonucleotides that are at least 5 nucleotides long. In some embodiments, the readout probe comprises oligonucleotides that are at least 6 nucleotides long. In some embodiments, the readout probe comprises oligonucleotides that are at least 7 nucleotides long. In some embodiments, the readout probe comprises oligonucleotides that are at least 8 nucleotides long. In some embodiments, the readout probe comprises oligonucleotides that are at least 9 nucleotides long. In some embodiments, the readout probe comprises oligonucleotides that are at least 10 nucleotides long. In some embodiments, the readout probe comprises oligonucleotides that are at least 11 nucleotides long. In some embodiments, the readout probe comprises oligonucleotides that are at least 12 nucleotides long. In some embodiments, the readout probe comprises oligonucleotides that are at least 13 nucleotides long. In some embodiments, the readout probe comprises oligonucleotides that are at least 14 nucleotides long. In some embodiments, the readout probe comprises oligonucleotides that are at least 15 nucleotides long. In some embodiments, the readout probe comprises oligonucleotides that are at least 16 nucleotides long. In some embodiments, the readout probe comprises oligonucleotides that are at least 17 nucleotides long. In some embodiments, the readout probe comprises oligonucleotides that are at least 18 nucleotides long. In some embodiments, the readout probe comprises oligonucleotides that are at least 19 nucleotides long. In some embodiments, the readout probe comprises oligonucleotides that are at least 20 nucleotides long. In some embodiments, the readout probe comprises oligonucleotides that are at least 21 nucleotides long. In some embodiments, the readout probe comprises oligonucleotides that are at least 22 nucleotides long. In some embodiments, the readout probe comprises oligonucleotides that are at least 23 nucleotides long. In some embodiments, the readout probe comprises oligonucleotides that are at least 24 nucleotides long. In some embodiments, the readout probe comprises oligonucleotides that are at least 25 nucleotides long. In some embodiments, the readout probe comprises oligonucleotides that are at least 26 nucleotides long. In some embodiments, the readout probe comprises oligonucleotides that are at least 27 nucleotides long. In some embodiments, the readout probe comprises oligonucleotides that are at least 28 nucleotides long. In some embodiments, the readout probe comprises oligonucleotides that are at least 29 nucleotides long. In some embodiments, the readout probe comprises oligonucleotides that are at least 30 nucleotides long. In some embodiments, the readout probes of any of the previous embodiments comprises oligonucleotides that are less than 35, 40, 45, 50, 100 nucleotides in length.

In some embodiments, the readout probe comprises a sequence flanking the 5′, 3′, or both the 5′ and 3′ end of the readout probe, wherein the sequence is complementary to the sample identifier region. In some embodiments, the sequence complementarity comprises at least 60%, 65%, 70%, 75%, 80%, 85%, 90%, 91%, 92%, 93%, 94%, 95%, 96%, 97%, 98%, 99%, or 100%.

### Intermediate Probes

In some embodiments, the method comprises sample readout probes interacting with their sample identifiers through one or more sample intermediate probes. In some embodiments, the method comprises target probes interacting with their targets through one or more target intermediate probes.

In some embodiments, a readout probe interacts with its target through binding or hybridization to one or more intermediate probe. In some embodiments, the intermediate probe comprises an oligonucleotide, antibody, antibody fragment, protein, or any combination thereof.

In some embodiments, the intermediate probe binds, hybridizes, or otherwise links to the target. In some embodiments, the method comprises a readout oligonucleotide interacting with a target through hybridization with an intermediate probe hybridized to a target, wherein the intermediate probe comprises a sequence complimentary to the target, and an overhang sequence. In some embodiments, the sequence complementarity comprises at least 60%, 65%, 70%, 75%, 80%, 85%, 90%, 91%, 92%, 93%, 94%, 95%, 96%, 97%, 98%, 99%, or 100%.

In some embodiments, the intermediate probe comprises an oligonucleotide. In some embodiments, the intermediate probe comprises an oligonucleotide that is at least 5 nucleotides long. In some embodiments, the intermediate probe comprises an oligonucleotide that is at least 6 nucleotides long. In some embodiments, the intermediate probe comprises an oligonucleotide that is at least 7 nucleotides long. In some embodiments, the intermediate probe comprises an oligonucleotide that is at least 8 nucleotides long. In some embodiments, the intermediate probe comprises an oligonucleotide that is at least 9 nucleotides long. In some embodiments, the intermediate probe comprises an oligonucleotide that is at least 10 nucleotides long. In some embodiments, the intermediate probe comprises an oligonucleotide that is at least 11 nucleotides long. In some embodiments, the intermediate probe comprises an oligonucleotide that is at least 12 nucleotides long. In some embodiments, the intermediate probe comprises an oligonucleotide that is at least 13 nucleotides long. In some embodiments, the intermediate probe comprises an oligonucleotide that is at least 14 nucleotides long. In some embodiments, the intermediate probe comprises an oligonucleotide that is at least 15 nucleotides long. In some embodiments, the intermediate probe comprises an oligonucleotide that is at least 16 nucleotides long. In some embodiments, the intermediate probe comprises an oligonucleotide that is at least 17 nucleotides long. In some embodiments, the intermediate probe comprises an oligonucleotide that is at least 18 nucleotides long. In some embodiments, the intermediate probe comprises an oligonucleotide that is at least 19 nucleotides long. In some embodiments, the intermediate probe comprises an oligonucleotide that is at least 20 nucleotides long. In some embodiments, the intermediate probe comprises an oligonucleotide that is at least 21 nucleotides long. In some embodiments, the intermediate probe comprises an oligonucleotide that is at least 22 nucleotides long. In some embodiments, the intermediate probe comprises an oligonucleotide that is at least 23 nucleotides long. In some embodiments, the intermediate probe comprises an oligonucleotide that is at least 24 nucleotides long. In some embodiments, the intermediate probe comprises an oligonucleotide that is at least 25 nucleotides long. In some embodiments, the intermediate probe comprises an oligonucleotide that is at least 26 nucleotides long. In some embodiments, the intermediate probe comprises an oligonucleotide that is at least 27 nucleotides long. In some embodiments, the intermediate probe comprises an oligonucleotide that is at least 28 nucleotides long. In some embodiments, the intermediate probe comprises an oligonucleotide that is at least 29 nucleotides long. In some embodiments, the intermediate probe comprises an oligonucleotide that is at least 30 nucleotides long. In some embodiments, the intermediate probes of any of the previous embodiments comprises oligonucleotides that are less than 35, 40, 45, 50, 100 nucleotides in length.

In some embodiments, the intermediate probe comprises an overhang sequence that is complementary to a readout probe. In some embodiments, the sequence complementarity comprises at least 60%, 65%, 70%, 75%, 80%, 85%, 90%, 91%, 92%, 93%, 94%, 95%, 96%, 97%, 98%, 99%, or 100%.

In some embodiments, the intermediate probe comprises an overhang sequence is complementary to a bridge probe. In some embodiments, the bridge probe comprises a sequence complementary to the readout probe. In some embodiments, the bridge probe comprises a sequence complementary to an intermediate probe.

In some embodiments, the method comprises intermediate probes that are preserved through multiple contacting and imaging steps. In some embodiments, the method comprises a removing step that removes readout probes, optionally keeping the intermediate probes intact. In some embodiments, the method comprises a removing step that removes the readout probes and keeps the intermediate probes intact. In some embodiments, readout probes differ from the intermediate probes in a chemical or enzymatic perspective, so that detectably labeled oligonucleotides can be selectively removed.

In some embodiments, the sample intermediate probe comprises a sequence complementary to the sample identifier and to the sample readout probe. In some embodiments, the sample intermediate probe is selected from proteins, modified proteins, RNA, oligonucleotides, antibodies, antibody fragments, and combinations thereof. In certain embodiments, the sample intermediate probe comprises an oligonucleotide.

In some embodiments, the sample intermediate probe comprises a sequence complementary to a sample identifier and a sequence complementary to a sample readout probe.

In some embodiments, the sample intermediate probe comprises a sequence complementary to a sample identifier and an overhang sequence. In some embodiments, the overhang sequence is complementary to a sample readout probe. In some embodiments, the overhang sequence is complementary to a sample bridge probe. In some embodiments, the sample bridge probe is complementary to a sample readout probe and to a sample intermediate probe intermediate probe. In some embodiments, the sample intermediate probes are preserved through multiple contacting and imaging steps.

In some embodiments, the target intermediate probe comprises a sequence complementary to the target and to the target readout probe. In some embodiments, the target intermediate probe is selected from proteins, modified proteins, RNA, oligonucleotides, antibodies, antibody fragments, and combinations thereof. In certain embodiments, each target intermediate probe comprises an oligonucleotide.

In some embodiments, the target intermediate probe comprises a sequence complementary to the target and to a target readout probe.

In some embodiments, the target probes interact with their targets through one or more target intermediate probes. In some embodiments, the target intermediate probes hybridize to targets. In some embodiments, the target intermediate probe comprises a sequence complementary to its target and an overhang sequence. In some embodiments, the overhang sequence is complementary to a target readout probe. In some embodiments, the overhang sequence is complementary to a target bridge probe. In some embodiments, each target bridge probe is complementary to a target readout probe and to an intermediate probe. In some embodiments, the target intermediate probes are preserved through multiple contacting and imaging steps

In some embodiments, the In some embodiments, the sequence complementarity of any of the previous embodiments comprises at least 60%, 65%, 70%, 75%, 80%, 85%, 90%, 91%, 92%, 93%, 94%, 95%, 96%, 97%, 98%, 99%, or 100%.

### Barcoding the Targets

In some embodiments, the method comprises barcoding one or more targets in the pooled sample. In some embodiments, the method comprises the targets that are selected from proteins, modified proteins, transcripts, RNA, DNA loci, exogenous proteins, exogenous nucleic acids, hormones, carbohydrates, small molecules, biologically active molecules, and combinations thereof. In some embodiments, the targets comprise subcellular features. For example, the nuclear lamin can be labeled with one set of barcodes, and nucleolus can be targeted with another set of barcodes. Then each sample can be uniquely labeled with a combination of barcodes on different subcellular compartments.

In some embodiments, the method comprises targets that are selected from nucleic acids or proteins involved in biosynthetic capacity, anaerobic physiology, stress responses, cellular signaling, biofilm matrix components, motility, all major quorum-sensing (QS) systems, multiple antibiotic resistance and core virulence factors.

In some embodiments, the method comprises targets that are selected form nucleic acids or proteins involved in biosynthetic capacity, anaerobic physiology, stress responses, cellular signaling, biofilm matrix components, motility, all major quorum-sensing (QS) systems, multiple antibiotic resistance and core virulence factors.

In some embodiments, the method comprises barcoding targets, wherein the targets are different.

In some embodiments, the method comprises fluorescence detection. In some embodiments, the method comprises fluorescence detection or other methods of detection. In some embodiments, the method comprises sequential hybridization to detect target analytes.

Certain techniques for analyzing the biological samples are known. See, for example, International PCT Patent Application No. PCT/US2014/036258, filed Apr. 30, 2014 and titled MULTIPLEX LABELING OF MOLECULES BY SEQUENTIAL HYBRIDIZATION BARCODING, the entire contents of which are herein incorporated by reference in its entirety for all purposes.

In some embodiments, the method comprises target probes that are selected from proteins, modified proteins, RNA, oligonucleotides, antibodies, antibody fragments, and combinations thereof.

In some embodiments, the method comprises contacting each sample in the one or more samples with a first plurality of target probes, so that the target probes interact with one or more targets.

In some embodiments, the method comprises contacting the target probes to the two or more samples before the two or more samples are pooled.

In some embodiments, the method comprises imaging the sample after the first contacting step so that interaction of the target probes with their targets is detected.

In some embodiments, the method comprises a contacting step that differs from another contacting step in the labelling of at least one of the targets.

In some embodiments, the method comprises a contacting step wherein each target probe in the first plurality of probes is labelled with a detectably moiety.

In some embodiments, the method comprises a contacting step wherein each target probe comprises a detectable moiety and at least one contacting step differs from another contacting step by having a different detectable moiety for each target.

In some embodiments, the method comprises a contacting step wherein at least two different target probes that interact with a first target and wherein at least two different target probes interact with a second target.

In some embodiments, the target probes comprise one or more labels selected from two, three, or four different labels.

In some embodiments, the barcode for the target in the sample comprises a signal that is amplified. In certain embodiments, the barcode for the target in a sample comprises a signal that is amplified by hybridization chain reaction, rolling circle amplification, or other amplification methods known to a person of skill.

In some embodiments, the method comprises using target probes wherein each target probe comprises the same detectable moiety and the same sequence.

In some embodiments, the method comprises target probes wherein each target probe interacts with its target through one or more target intermediate probes each of which is hybridized to the target.

In some embodiments, the target probes of any of the previous embodiments are detectably labelled. In some embodiments, the target probes hybridize to the targets. In some embodiments, the target probes are target readout probes.

In some embodiments, the method comprises repeating the contacting and imaging steps, each time with a new plurality of target probes so that a target in the sample is described by a barcode, and can be differentiated from another target in the sample by a difference in their barcodes.

In some embodiments, the method comprises an error correction round performed by selecting from block codes such as Hamming codes, Reed-Solomon codes, Golay codes, or any combination thereof.

### Demultiplexing the Sample

In some embodiments, the method comprises demultiplexing the sample probes to associate cells with their samples.

In some embodiments, demultiplexing comprises imaging the pooled sample after the first contacting step so that interaction of the sample probes with their sample identifiers is detected. In some embodiments, demultiplexing comprises repeating the contacting and imaging steps, each time with a new plurality of sample probes so that a barcode is created for each sample, and can be differentiated from sample cells in the other samples by a difference in their barcodes.

In some embodiments, the method comprises demultiplexing the sample probes to associate cells with their samples after repeating the contacting and imaging steps, each time with a new plurality of sample probes so that a sample is described by a barcode, and can be differentiated from another sample by a difference in their barcodes.

In certain embodiments, demultiplexing comprises analyzing background and signals generated by the sample probes interacting with the sample identifiers, the sample identifiers interacting with the sample identifiers within segmented boundaries to provide a signal-to-background score for each readout. In certain embodiments, the demultiplexing comprises classifying the cells according to the positive readout signals.

### Removing Probes

In some embodiments, the method comprises a step of removing the one or more probes after one or more imaging steps. In some embodiments, the step of removing the probes comprises contacting the plurality of readout probes with an enzyme that digests the probes. In some embodiments, the step of removing comprises contacting the plurality of probes with a DNase, contacting the plurality of probes with an RNase, photobleaching, strand displacement, formamide wash, heat denaturation, chemical denaturation, cleavage, or combinations thereof. In some embodiments, the step of removing comprises photobleaching to remove the probes.

In some embodiments, the method further comprises comprising removing the readout probes after one or more imaging steps. In some embodiments, the method comprises the step of removing comprises contacting the plurality of readout probes with an enzyme that digests a readout probe. In some embodiments, the method comprises removing the readout probes by using stripping reagents, wash buffers, photobleaching, chemical bleaching, and any combinations thereof. In some embodiments, the method comprises contacting the plurality of target readout probes with a DNase, contacting the plurality of target probes with an RNase, photobleaching, strand displacement, formamide wash, heat denaturation, or combinations thereof. In some embodiments, the target readout probes are removed by photobleaching.

In some embodiments, the method comprises clearing the sample. In some embodiments the sample is cleared by CLARITY. In some embodiments, the sample is cleared following hydrogel embedding.

Certain techniques for removing probes are known in the art. See, for example, International PCT Patent Application No. PCT/US2014/036258, filed Apr. 30, 2014 and titled MULTIPLEX LABELING OF MOLECULES BY SEQUENTIAL HYBRIDIZATION BARCODING, the entire contents of which are herein incorporated by reference in its entirety for all purposes.

### Imaging the Sample

In some embodiments, the method comprises imaging the sample probes or barcodes. In some embodiments, the method comprises imaging the target probes or barcodes. As understood by a person having ordinary skill in the art, different technologies can be used for the imaging steps.

In some embodiments, the imaging methods comprise but are not limited to epi-fluorescence microscopy, confocal microscopy, the different types of super-resolution microscopy (PALM/STORM, SSIM/GSD/STED), and light sheet microscopy (SPIM and etc).

In some embodiments, the imaging methods comprise exemplary super resolution technologies include, but are not limited to I⁵M and 4Pi-microscopy, Stimulated Emission Depletion microscopy (STEDM), Ground State Depletion microscopy (GSDM), Spatially Structured Illumination microscopy (SSIM), Photo-Activated Localization Microscopy (PALM), Reversible Saturable Optically Linear Fluorescent Transition (RESOLFT), Total Internal Reflection Fluorescence Microscope (TIRFM), Fluorescence-PALM (FPALM), Stochastical Optical Reconstruction Microscopy (STORM), Fluorescence Imaging with One-Nanometer Accuracy (FIONA), and combinations thereof. For examples: Chi, 2009 “Super-resolution microscopy: breaking the limits,” Nature Methods 6(1): 15-18; Blow 2008, “New ways to see a smaller world,” Nature 456:825-828; Hell, et al, 2007, “Far-Field Optical Nanoscopy,” Science 316: 1153; R. Heintzmann and G. Ficz, 2006, “Breaking the resolution limit in light microscopy,” Briefings in Functional Genomics and Proteomics 5(4):289-301; Garini et al., 2005, “From micro to nano: recent advances in high-resolution microscopy,” Current Opinion in Biotechnology 16:3-12; and Bewersdorf et al, 2006, “Comparison of I⁵M and 4Pi-microscopy,” 222(2): 105-1 17; and Wells, 2004, “Man the Nanoscopes,” JCB 164(3):337-340.

In some embodiments, electron microscopes (EM) are used for imaging.

In some embodiments, an imaging step detects a target. In some embodiments, an imaging step localizes a target. In some embodiments, an imaging step provides three-dimensional spatial information of a target. In some embodiments, an imaging step quantifies a target. By using multiple contacting and imaging steps, provided methods are capable of providing spatial and/or quantitative information for a large number of targets in surprisingly high throughput. For example, when using F detectably different types of labels, spatial and/or quantitative information of up to FN targets can be obtained after N contacting and imaging steps.

Certain techniques for imaging are known in the art. See, for example, International PCT Patent Application No. PCT/US2014/036258, filed Apr. 30, 2014 and titled MULTIPLEX LABELING OF MOLECULES BY SEQUENTIAL HYBRIDIZATION BARCODING, the entire contents of which are herein incorporated by reference in its entirety for all purposes.

In some embodiments, the method comprises analyzing cell size and shape, markers, immunofluorescence measurements, or any combinations thereof.

### Flurophores

In some embodiments, the method comprises detecting the probes, readout probes, or oligonucleotides thereof with fluorophores. In some embodiments, the probes of any of the previous embodiments comprises a fluorophore.

In some embodiments, the fluorophore is any fluorophore deemed suitable by those of skill in the arts.

In some embodiments, the fluorophores include but are not limited to fluorescein, rhodamine, Alexa Fluors, DyLight fluors, ATTO Dyes, or any analogs or derivatives thereof. In certain embodiments, the detectable moieties include but are not limited to fluorescein and chemical derivatives of fluorescein; Eosin; Carboxyfluorescein; Fluorescein isothiocyanate (FITC); Fluorescein amidite (FAM); Erythrosine; Rose Bengal; fluorescein secreted from the bacterium Pseudomonas aeruginosa; Methylene blue; Laser dyes; Rhodamine dyes (e.g., Rhodamine, Rhodamine 6G, Rhodamine B, Rhodamine 123, Auramine O, Sulforhodamine 101, Sulforhodamine B, and Texas Red).

In some embodiments, the fluorphores include but are not limited to ATTO dyes; Acridine dyes (e.g., Acridine orange, Acridine yellow); Alexa Fluor; 7-Amino actinomycin D; 8-Anilinonaphthalene-1-sulfonate; Auramine-rhodamine stain; Benzanthrone; 5,12-Bis(phenylethynyl) naphthacene; 9,10-Bis(phenylethynyl)anthracene; Blacklight paint; Brainbow; Calcein; Carboxyfluorescein; Carboxyfluorescein diacetate succinimidyl ester; Carboxyfluorescein succinimidyl ester; 1-Chloro-9,10-bis(phenylethynyl)anthracene; 2-Chloro-9,10-bis(phenyl ethynyl)anthracene; 2-Chloro-9,10-diphenylanthracene; Coumarin; Cyanine dyes (e.g., Cyanine such as Cy3 and Cy5, DiOC6, SYBR Green I); DAPI, Dark quencher, DyLight Fluor, Fluo-4, FluoProbes; Fluorone dyes (e.g., Calcein, Carboxyfluorescein, Carboxyfluorescein diacetate succinimidyl ester, Carboxyfluorescein succinimidyl ester, Eosin, Eosin B, Eosin Y, Erythrosine, Fluorescein, Fluorescein isothiocyanate, Fluorescein amidite, Indian yellow, Merbromin); Fluoro-Jade stain; Fura-2; Fura-2-acetoxymethyl ester; Green fluorescent protein, Hoechst stain, Indian yellow, Indo-1, Lucifer yellow, Luciferin, Merocyanine, Optical brightener, Oxazin dyes (e.g., Cresyl violet, Nile blue, Nile red); Perylene; Phenanthridine dyes (Ethidium bromide and Propidium iodide); Phloxine, Phycobilin, Phycoerythrin, Phycoerythrobilin, Pyranine, Rhodamine, Rhodamine 123, Rhodamine 6G, RiboGreen, RoGFP, Rubrene, SYBR Green I, (E)-Stilbene, (Z)-Stilbene, Sulforhodamine 101, Sulforhodamine B, Synapto-pHluorin, Tetraphenyl butadiene, Tetrasodium tris(bathophenanthroline disulfonate) ruthenium(II), Texas Red, TSQ, Umbelliferone, or Yellow fluorescent protein.

In some embodiments, the fluorophores include but are not limited to Alexa Fluor family of fluorescent dyes (Molecular Probes, Oregon). Alexa Fluor dyes are widely used as cell and tissue labels in fluorescence microscopy and cell biology. The excitation and emission spectra of the Alexa Fluor series cover the visible spectrum and extend into the infrared. The individual members of the family are numbered according roughly to their excitation maxima (in nm). Certain Alexa Fluor dyes are synthesized through sulfonation of coumarin, rhodamine, xanthene (such as fluorescein), and cyanine dyes. In some embodiments, sulfonation makes Alexa Fluor dyes negatively charged and hydrophilic. In some embodiments, Alexa Fluor dyes are more stable, brighter, and less pH-sensitive than common dyes (e.g. fluorescein, rhodamine) of comparable excitation and emission, and to some extent the newer cyanine series. Exemplary Alexa Fluor dyes include but are not limited to Alexa-350, Alexa-405, Alexa-430, Alexa-488, Alexa-500, Alexa-514, Alexa-532, Alexa-546, Alexa-555, Alexa-568, Alexa-594, Alexa-610, Alexa-633, Alexa-647, Alexa-660, Alexa-680, Alexa-700, or Alexa-750.

In some embodiments, the fluorophores comprise one or more of the DyLight Fluor family of fluorescent dyes (Dyomics and Thermo Fisher Scientific). Exemplary DyLight Fluor family dyes include but are not limited to DyLight-350, DyLight-405, DyLight-488, DyLight-549, DyLight-594, DyLight-633, DyLight-649, DyLight-680, DyLight-750, or DyLight-800.

In some embodiments, the fluorophore comprises a nanomaterial. In some embodiments, the fluorophore is a nanoparticle. In some embodiments, the fluorophore is or comprises a quantum dot. In some embodiments, the fluorophore is a quantum dot. In some embodiments, the fluorophore comprises a quantum dot. In some embodiments, the fluorophore is or comprises a gold nanoparticle. In some embodiments, the fluorophore is a gold nanoparticle. In some embodiments, the fluorophore comprises a gold nanoparticle.

### Washes

In some embodiments, the method of any of the preceding embodiments, comprises optionally washing the sample after each step. In certain embodiments, the sample is washed with a buffer that removes non-specific hybridization reactions. In certain embodiments, formamide is used in the wash step. In certain embodiments, the wash buffer is stringent. In certain embodiments, the wash buffer comprises 10% formamide, 2×SSC, and 0.1% triton X-100s.

Having described the embodiments in detail, it will be apparent that modifications, variations, and equivalent embodiments are possible without departing the scope of the defined in the appended claims. Furthermore, it should be appreciated that all examples in the present disclosure are provided as non-limiting examples.

The following non-limiting methods and examples are provided to further illustrate the embodiments disclosed herein. It should be appreciated by those of skill in the art that the techniques disclosed in the methods and examples that follow represent approaches that have been found to function well in practice, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the embodiments.

### Examples

The following methods and experiments utilize the embodiments of the methods as previously described, to analyze Pseudomonas aeruginosa planktonic and biofilm populations to demonstrate how different cellular functions are coordinated in time and space.

The following methods provide proof-of-concept work illustrates how the ability to observe transcriptional activities at the microscale permits insights into the spatiotemporal regulation and coordination of critical life processes, enabling hitherto unrecognized, transient physiological states to be identified and new hypotheses to be generated.

### Method 1

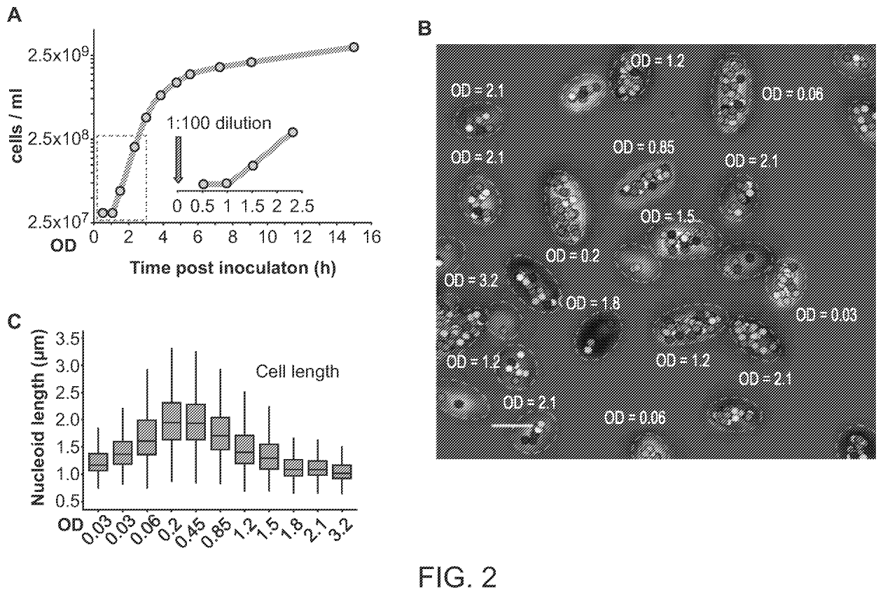

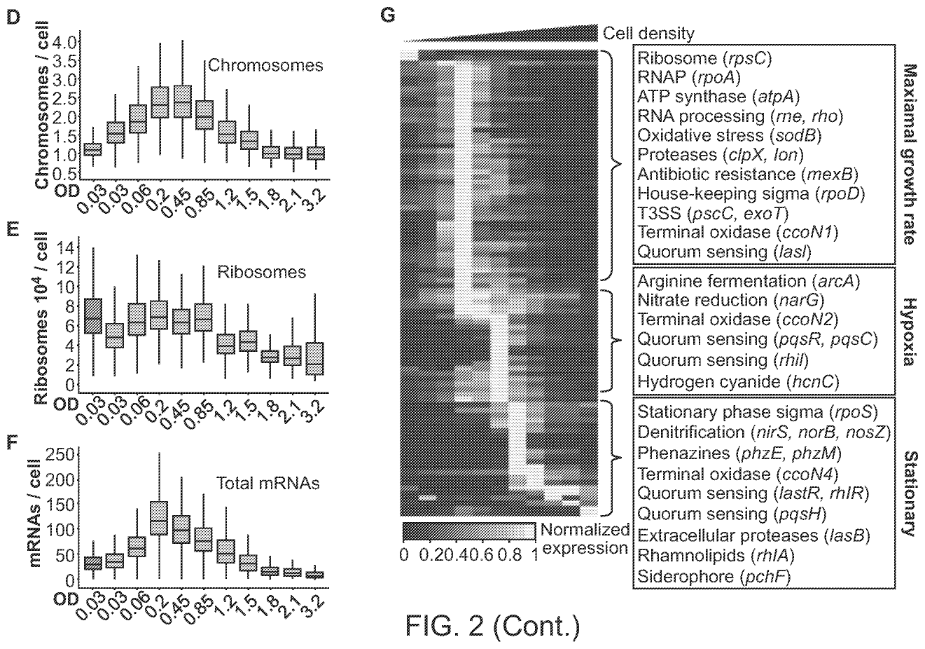

P. aeruginosa strain UCBPP-PA14 was grown aerobically with shaking at 250 rpm in lysogeny broth (LB) (Difco) or on LB agar plates at 37° C. SCFM was made as previously described (Palmer et al., 2007). For the growth curve experiments, an overnight LB culture was washed twice using fresh growth media (either LB or SCFM) and then diluted 1:100 into 100 ml pre-warmed fresh media. The cultures were grown at 37° C. with shaking at 250 rpm and collected at various time points as indicated in FIG. 2A. The SCFM samples were collected cell densities identical to the LB experiment, except the OD600=3.2 sample was ommited. Collected samples were immediately fixed in ice-cold 2% paraformaldehyde (PFA) and were incubated on ice for 1.5 h in the dark, and then washed twice with lx PBS. Samples were resuspended in 70% EtOH and incubated at −20° C. for at 24 h to permeabilize the cells. Surface colonization was performed by washing and diluting an LB overnight culture 1:100 into fresh SCFM and dispensing 100 μl into coverslip attached open incubation chambers (Electron Microscopy Sciences, 70333-42). The coverslips were incubated in parafilm sealed sterile petri dishes at 37° C. and the media was gently exchanged every 4 hours. A damp Kimwipe was placed in the petri dish to control media evaporation. During the overnight stage of the 35 h experiment, the media was exchanged only once after 8 h. Biofilm experiments were collected by gently exchanging the SCFM with 100 μl ice cold 2% PFA solution and incubating the sample at 4° C. for 1.5 h. The samples were washed twice with lx PBS, resuspended in 70% EtOH and incubated overnight at 4° C. and prepared for seqFISH as described below the following day.

### Method 2

Primary probes were designed as 30 nt stretches in a GC range of 45-65%. Probe sequences containing more than four consecutive base repeats were removed. The remaining probes were compared to the reference genome using blast and any probe with non-specific binding of at 18 nt or more was discarded. Negative control genes were selected from the P1 phage genome sequence (NC_005856.1) with the same criteria as above. Each selected gene was covered by 12-20 nonoverlapping probes randomly selected from the gene probe set. The probes were designed as a 30 nt mRNA binding region flanked by overhangs composed of four repeats of the secondary hybridization sequence (complementary to a designated fluorescent readout probe; See Table S2 of U.S. Provisional Patent Application No. 63/153,234, filed Feb. 24, 2021 incorporated by its entirety).

Thus, it is estimated that during secondary hybridization, each mRNA is covered by 48-80 fluorescent readout probes (12-20×4), on par with previous mRNA-FISH experiments in bacteria (Skinner et al., 2013; So et al., 2011).

A library of 1,764 probes targeting 105 P. aeruginosa genes and three negative controls was designed (See Tables S1-S2 of U.S. Provisional Patent Application No. 63/153,234, filed Feb. 24, 2021 incorporated by its entirety). Additional flanking sequences were added to the primary probe sequences to enable library amplification via PCR (Forward 5′-TTTCGTCCGCGAGTGACCAG-3′ and reverse 5′-CAACGTCCATGTCGGGATGC-′). The primary probe set was purchased as oligoarray complex pool from Twist Bioscience and constructed as previously described (Eng et al., 2019) (Table S2). Briefly, a set of 9 PCR cycles were used to amplify the designated probe sequences from the oligo pool. The amplified PCR products were purified using the QIAquick PCR Purification Kit (28104; Qiagen) according to the manufacturer's instructions. The PCR products were used as the template for in vitro transcription (E2040S; NEB) followed by reverse transcription (EP7051; Thermo Fisher).

Then, the single-stranded DNA (ssDNA) probes were alkaline hydrolysed with 1 M NaOH at 65° C. for 15 min to degrade the RNA templates, followed by 1 M acetic acid neutralization. Next, to clean up the probes, we performed ethanol precipitation to remove stray nucleotides, phenol-chloroform extraction to remove protein, and used Zeba Spin Desalting Columns (7K MWCO) (89882; Thermo Fisher) to remove residual nucleotides and phenol contaminants. Readout probes were designed as previously described and ordered from Integrated DNA Technologies (IDT) (Eng et al., 2019).

Ribo-Tag probes were designed to target the same region in the 16S rRNA gene according to the criteria described above, but with a 28 nt binding regions. Each probe sequence was flanked with two secondary sequences selected out a set of six that were dedicated to multiplexing (Table 1).

TABLE 1
Exemplary 16S rRNA probes

| Probe_id | 16S pos | 16S seq | Full probe sequence |
|---|---|---|---|
| Ribo-Tag_1 | 771-799 | GCGTGGACTACCA | CGATTAGTCGTCACTAAG |
| | | GGGTATCTAATCCT | CGTGGACTACCAGGGTAT |
| | | G | CTAATCCTGAAACTCCGA |
| | | | ATGCTACG |
| Ribo-Tag_2 | 771-799 | GCGTGGACTACCA | CGATTAGTCGTCACTAAG |
| | | GGGTATCTAATCCT | CGTGGACTACCAGGGTAT |
| | | G | CTAATCCTGAAGGTTACA |
| | | | CGCGACTA |
| Ribo-Tag_3 | 771-799 | GCGTGGACTACCA | ACTCCGAATGCTACGAAG |
| | | GGGTATCTAATCCT | CGTGGACTACCAGGGTAT |
| | | G | CTAATCCTGAAGGTTACA |
| | | | CGCGACTA |
| Ribo-Tag_4 | 771-799 | GCGTGGACTACCA | ATGTAACCAAGCGTCAAG |
| | | GGGTATCTAATCCT | CGTGGACTACCAGGGTAT |
| | | G | CTAATCCTGAATCAGTTA |
| | | | CCGGTGTA |
| Ribo-Tag_5 | 771-799 | GCGTGGACTACCA | ATGTAACCAAGCGTCAAG |
| | | GGGTATCTAATCCT | CGTGGACTACCAGGGTAT |
| | | G | CTAATCCTGAATCCAGCT |
| | | | TACGTTCG |
| Ribo-Tag_6 | 771-799 | GCGTGGACTACCA | TCAGTTACCGGTGTAAAG |
| | | GGGTATCTAATCCT | CGTGGACTACCAGGGTAT |
| | | G | CTAATCCTGAATCCAGCT |
| | | | TACGTTCG |
| Ribo-Tag_7 | 771-799 | GCGTGGACTACCA | CGATTAGTCGTCACTAAG |
| | | GGGTATCTAATCCT | CGTGGACTACCAGGGTAT |
| | | G | CTAATCCTGAATCAGTTA |
| | | | CCGGTGTA |
| Ribo-Tag_8 | 771-799 | GCGTGGACTACCA | CGATTAGTCGTCACTAAG |
| | | GGGTATCTAATCCT | CGTGGACTACCAGGGTAT |
| | | G | CTAATCCTGAATCCAGCT |
| | | | TACGTTCG |
| Ribo-Tag_9 | 771-799 | GCGTGGACTACCA | ATGTAACCAAGCGTCAAG |
| | | GGGTATCTAATCCT | CGTGGACTACCAGGGTAT |
| | | G | CTAATCCTGAAACTCCGA |
| | | | ATGCTACG |
| Ribo- | 771-799 | GCGTGGACTACCA | ATGTAACCAAGCGTCAAG |
| Tag_10 | | GGGTATCTAATCCT | CGTGGACTACCAGGGTAT |
| | | G | CTAATCCTGAAGGTTACA |
| | | | CGCGACTA |
| Ribo- | 771-799 | GCGTGGACTACCA | ACTCCGAATGCTACGAAG |
| Tag_11 | | GGGTATCTAATCCT | CGTGGACTACCAGGGTAT |
| | | G | CTAATCCTGAATCCAGCT |
| | | | TACGTTCG |
| Ribo- | 771-799 | GCGTGGACTACCA | TCAGTTACCGGTGTAAAG |
| Tag_12 | | GGGTATCTAATCCT | CGTGGACTACCAGGGTAT |
| | | G | CTAATCCTGAAGGTTACA |
| | | | CGCGACTA |
| Reference | 714-743 | CAGTGTCAGTATCA | TATGTGACTACGCACAAC |
| (all | | GTCCAGGTGGTCGC | GTCGTATCCAGTATAACA |
| samples) | | CT | GTGTCAGTATCAGTCCAG |
| | | | GTGGTCGCCTAACTATTA |
| | | | TCGCCGAGA |

An additional 16S rRNA probe was generated as a standard between all multiplexed samples as was hybridized to an independent region of the 16S rRNA (Table 1)

### Method 3

### Coverslip Functionalization

Coverslips were cleaned with a plasma cleaner on a high setting (PDC-001, Harrick Plasma) for 5 min, followed by immersion in 1% bind-silane solution (GE; 17-1330-01) made in pH 3.5 10% (v/v) acidic ethanol solution for 30 min at room temperature. The coverslips were washed with 100% ethanol three times and dried in an oven at >90° C. for 30 min. The coverslips were then treated with 100 μg μl⁻¹ of poly-D-lysine (P6407; Sigma) in water for at least one hour at room temperature, followed by three rinses with water. Coverslips were air-dried and kept at −20° C. for no longer than 2 weeks before use.

### Method 4

### Parallel seqFISH

Independent fixed samples were individually hybridized with 16S rRNA labels, washed and then pooled into a single mixture that was hybridized with the gene probe library and prepared for imaging as described below. Approximately 108 cells were collected from each sample into a microcentrifuge, pelleted via centrifugation (6,000 rpm) and then resuspended in 20 μl H₂O with 6 nM of the designated 16S rRNA label (sample specific) and another 6 nM of a shared reference 16S rRNA probe (See Table S3: U.S. Provisional Patent Application No. 63/153,234, filed Feb. 24, 2021).

Each sample was then mixed with prewarmed 30 μl of primary hybridization buffer (50% formamide, 10% dextran sulfate and 2×SSC) via gentle pipetting, incubated at 37° C. for >16 h, washed twice with 100 μl wash buffer (55% formamide and 0.1% Triton-X 100 in 2×SSC; 5 min 8,000 rpm for the viscous hybridization buffer) and then incubated at 37° C. in 100 μl wash buffer for 30 min to remove non-specific probe binding. Samples were washed twice with 100 μl 2×SSC and pooled together into a new microcentrifuge in equal volumes. The mixture was pelleted and resuspended in 40 μl H₂O and 10 μl of the mixture was added to 10 μl gene probe library mixture and mixed well with prewarmed 30 μl primary hybridization buffer. The hybridizations were incubated for >16 h at 37° C. and were washed and prepared as described above. The final mixture was resuspended in 20-25 μl lx PBS and 5-10 μl were gently spotted at the center of the coverslip and incubated at RT for 10 min to allow the cells to sediment and bind the surface. The coverslips were centrifuged for 5 min at 1,000 rpm to create a smooth and dense cell monolayer. The cells were immobilized using a hydrogel as previously described (Eng et al., 2019) and stained with 10 μg m1-1 DAPI (D8417; Sigma) for 5 min before imaging so that cells could be visualized.

In biofilm experiments, the fixed and permeabilized surface attached microaggregates were air dried, covered with a hydrogel and hybridized with the gene library and a single rRNA probe in one single reaction, as described above.

All seqFISH experiments were performed using a combined imaging and automated fluidics delivery system as previously described (Eng et al., 2019). DAPI stained samples mounted on coverslips were connected to the fluidic system. The ROIs were registered using the DAPI fluorescence and a set of sequential secondary hybridizations, washes and imaging was performed as described below.

Each hybridization round contained three unique 15-nt readouts probes each conjugated to either Alexa Fluor 647 (A647), Cy3B and Alexa Fluor 488 (A488). All readout probes were ordered from Integrated DNA Technologies and prepared into 500 nM stock solutions. Each serial probe mixture was prepared in EC buffer (10% ethylene carbonate (E26258; Sigma), 10% dextran sulfate (D4911; Sigma), 4×SSC).

Hybridizations were incubated with the sample for 20 min to allow for secondary probe binding. The samples were then washed to remove excess readout probes and to limited non-specific binding using ˜300 μl of 10% formamide wash buffer (10% formamide and 0.1% Triton X-100 in 2×SSC). Samples were then rinsed with −200 μl of 4×SSC and then stained with DAPI solution (10 μg ml-1 of DAPI, 4×SSC). Lastly, an anti-bleaching buffer solution made was flowed through the samples (10% (w/v) glucose, 1:100 diluted catalase (Sigma C3155), 0.5 mg ml-1 glucose oxidase (Sigma G2133) and 50 mM pH 8 Tris-HCl in 4×SSC). Imaging was performed with a Leica DMi8 microscope equipped with a confocal scanner unit (Yokogawa CSU-W1), a sCMOS camera (Andor Zyla 4.2 Plus), a 63× oil objective lens (Leica 1.40 NA) and a motorized stage (ASI MS2000). Lasers from CNI and filter sets from Semrock were used. Snapshots were acquired using 647-nm, 561-nm, 488-nm and 405-nm fluorescent channels with 0.5-μm z-steps for all experiments with the exception of the 35 h biofilm experiment in which 1.0-μm z-steps were collected. After imaging, readout probes were stripped using 55% wash buffer (55% formamide and 0.1% Triton-X 100 in 2×SSC) that was flowed through for 1 min, followed by an incubation time of 15 min before rinsing with 4×SSC solution. This protocol: serial hybridizations, imaging and signal quenching steps, was repeated for −40 rounds to capture 16S rRNA for multiplexing, mRNA expression and background signal. The integration of automated fluidics delivery system and imaging was controlled via μManager (Edelstein et al., 2010).

### Method 5

### Image Analysis Demultiplexing and Gene-Expression Measurement

Maximal projection images were generated using ImageJ (Schneider et al., 2012) for DAPI and 16S rRNA and hybridization rounds were registered using the DAPI fluorescence. Aberrations between fluorophores were corrected by alignment of 16S rRNA signals across all channels. Cells were segmented using the DAPI signal with SuperSegger using the 60×Pa configuration (Stylianidou et al., 2016) and filtered using custom scripts to eliminate odd shapes, autofluorescent or low signal components.

For par-seqFISH demultiplexing, the background (no readouts) and 16S rRNA fluorescent intensity for each relevant secondary readout probe was measured within segmented cell boundaries to provide a signal-to-background score for each readout. The cells were classified according to the positive readout combinations (Table 1). The level of false positives was estimated by counting the number of cells classified into combinations left out of the experiment.

The mRNA-FISH data was analyzed using Spatzcells (Skinner et al., 2013). Briefly, spots were detected as regional maxima with intensity greater than a threshold value that was set using the negative control genes and were fit with a 2D gaussian model. The integrated intensity of the spot and the position of its estimated maxima were determined (Skinner et al., 2013). Spots were assigned to cells using the cell segmentation masks (Skinner et al., 2013). In biofilm experiments spots were assigned to cells in a z-section sensitive manner. Deviating spots maxima positions that did not overlap a cell boundary were tested against the flanking z-sections to identify their cell of origin. If no cell was detected the spots were discarded. All predicted low expression genes (defined as gene with spots in less than 30% of all cells) were identified and the distribution of their spot intensities was fit with a gaussian mixture model to identify the characteristic intensity of a single mRNA, normalized to the number of probes used for the specific gene. The median characteristic single-mRNA signal was then calculated using all low expression genes for each fluorophore (A647, A488 and cy3B). The variation between different genes labeled with the same fluorophore was low, with a coefficient of variation of 18-21%. This median characteristic value was used to transform fluorescent intensity into to discrete mRNA counts per gene within each cell. The A488 characteristic signal was corrected by a factor of 1.5 to account for its lower intensity in our system. In each cell, the total intensity of each gene was calculated by summing the intensities of all spots. The total value was normalized by the characteristic value for a single mRNA in the corresponding fluorophore.

### Method 6

### Single-Cell Expression Analysis and Cell Biological Parameter Calculations

Single-cell UMAP analysis was performed using Scanpy v1.7.0 (Wolf et al., 2018). Genes detected at consistently low levels were excluded from the analysis. These included pilY1, flgK, nasA, algU, purF, phzH, phzS and pslG (See Table 2: U.S. Provisional Patent Application No. 63/153,234, filed Feb. 24, 2021).

Standard Scanpy normalization and scaling, dimensionality reduction, and clustering as described in the Scanpy tutorial, minus the high variance gene selection stage. We used 15 neighbors and 15 and 17 PCA components, for the LB and SCFM experiments, respectively. Clustering was performed using the Leiden method. Jupyter notebooks with chosen parameters, run lines, output files and source data are available at github.com/daniedar/seqFISH.

Cell nucleoid size was calculated using the segmentation mask. A chromosome score was calculated as the median DAPI intensity multiplied by the nucleoid size. The median chromosome score was calculated for the last time point in our LB experiment (deep stationary; OD600=3.2). Since most cells in this stage are in a non-dividing state, we set this value as a reference for a single chromosome copy. The scores of all the cells were then normalized in the experiment using this value, as seen in FIG. 2. In addition to using Ribo-Tags to label cells from different conditions, we also hybridized another region in the 16S rRNA with a probe that was shared across all samples (Table 1). This reference signal was used to compare the 16S rRNA intensity between cells from different conditions. The median 16S rRNA signal per cells was measured and multiplied it by the nucleoid size (which completely overlaps the 16S signal and estimates cell size). In E. coli, maximal ribosome numbers appear at the maximal growth rate and have been estimated at 72,000 (Milo et al., 2010). The median rRNA score was calculated for the maximal growth (OD600=0.2) and normalized to 72,000 as in E. coli for a rough estimate (FIG. 2).

### Method 7

### Image Analysis in Surface Colonization Experiments

Images were registered as described above and segmentation was performed using the pixel classification workflow in Ilastik (Berg et al., 2019). Ilastik classification model was trained with background, cell boundaries and cell bodies, using the 16S rRNA signal.

However, in high density regions, segmentation often resulted in over-connectivity due to incorrect 3D overlaps. These cell clusters were disconnected. The binary masks (segmentation output) were thinned, and all 3D connected components (CCs) were re-calculated, reducing spurious connections. Then, all CCs traversing more than 2.5 μm were set aside for re-evaluation for potential over-connections. For each such 3D component we examined each z-slice at a time and identified all 2D CCs. Overly large or curved blobs were removed, which represent segmentation artefacts that often incorrectly connect distinct cells across zsections.

In addition, for each 2D detected component its orientation and overlap with components in the previous flanking z-section were calculated. If this component exhibited a significant change in its orientation (the direction it is pointing) it was disconnected from the component below. The analysis was continued using the newly oriented component as a seed. Cell clusters that could not be properly disentangled were removed from the analysis. At the end of the analysis the cell 3D masks were rethickened.

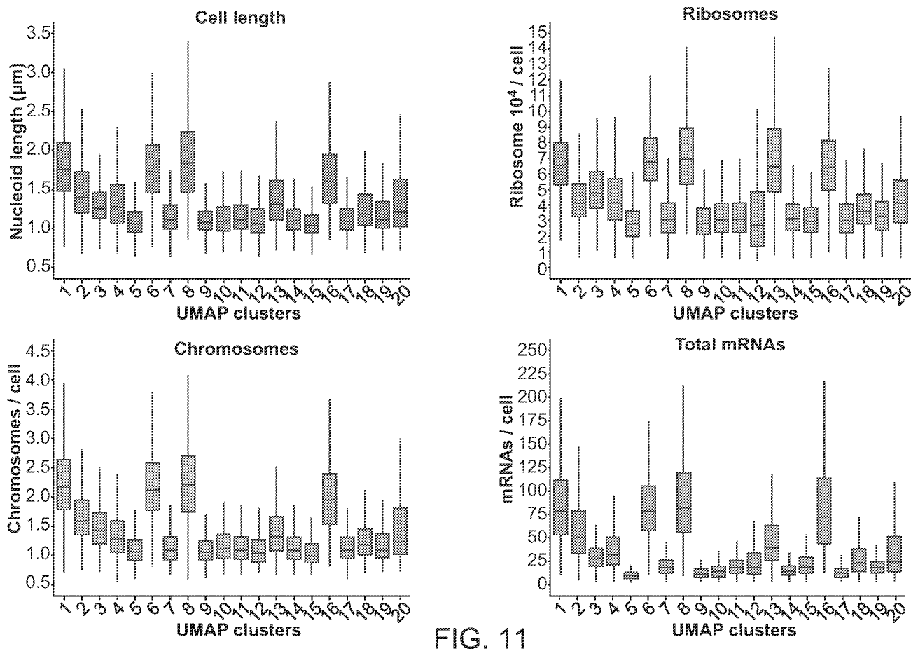

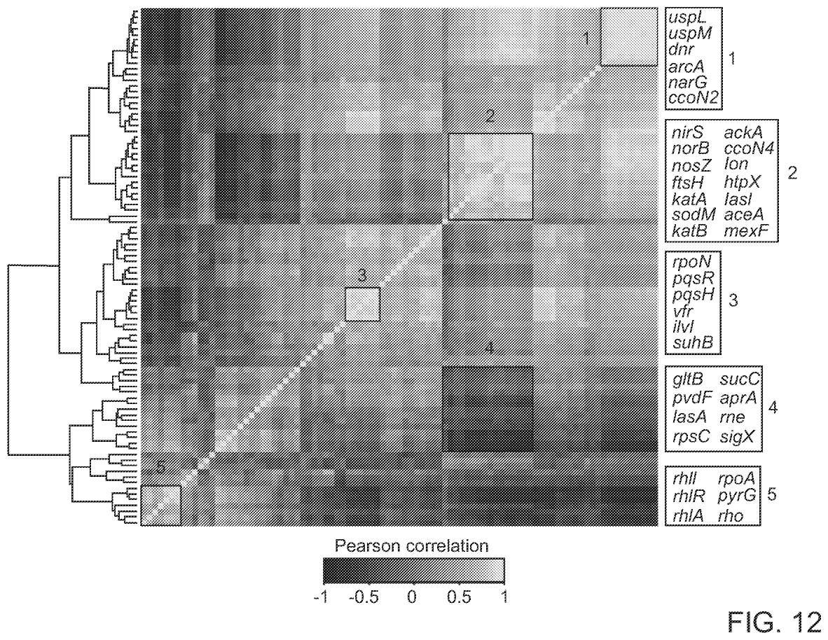

The bulk neighborhood analysis was conducted where we studied the immediate neighborhoods associated with high expression of a specific gene. For the gene of interest, we identified all top 99th percentile cells (99.5th for the pyocin specific analysis), denoted as “center cells”. Using the 3D centroid coordinates of center cells we identified their closest neighbors within a specified distance (up to 10 μm for pyocins and 3 μm for the rest). Up to k closest cells (up to 5-300 neighbors in the pyocin analysis to view the enrichment decay and up to 5 for the rest of the genes) were collected. All of the neighborhood cells selected (not including the center cells) were then analyzed in bulk together and their mean gene-expression was calculated and compared to the population (minus all center cells not used). This analysis was conducted across all genes and performed a Pearson correlation analyses to identify spatially correlating genes (FIG. 12).

### Experiment 1

### Parallel and Sequential mRNA-Fish in Single Bacterial Cells

To test our bacterial seqFISH approach, we first studied P. aeruginosa grown in well-understood batch culture conditions. We performed a growth curve experiment in LB medium, where key parameters such as cell density, growth rate, and oxygen levels change in a predictable manner. We collected 11 time points representing the lag phase, exponential growth, and stationary phase and imaged them simultaneously (FIG. 2A). Independent imaging of these samples in a serial manner would have taken ˜3 weeks of automated microscopy time.

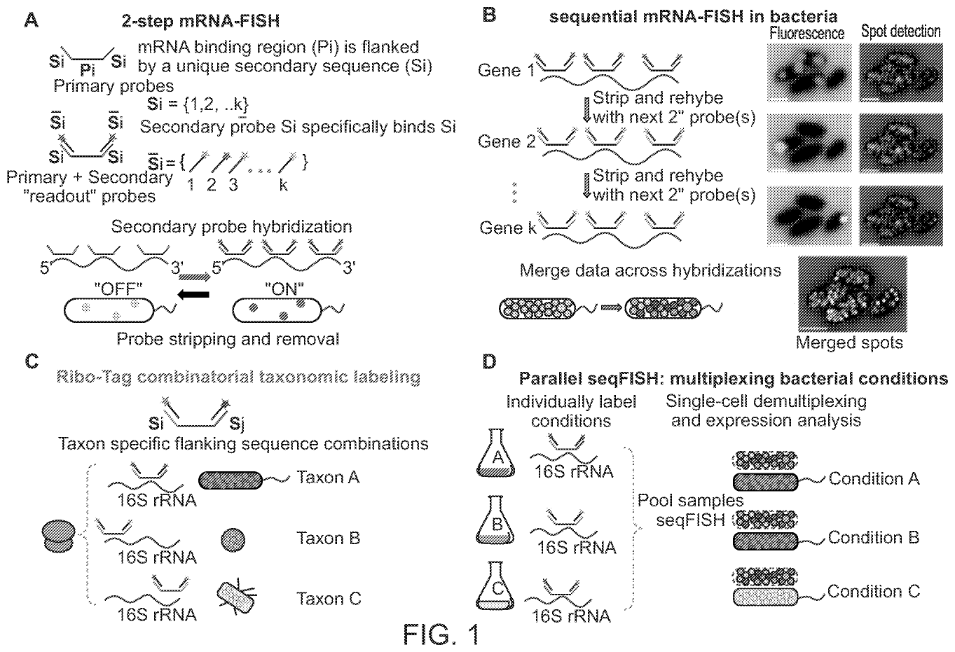

To perform simultaneous imaging, we developed an efficient multiplexing method that enables parallel seqFISH experiments (par-seqFISH). We designed a set of primary probes targeting the 16S rRNA (Ribo-Tags), which contain unique combinations of flanking sequences (barcodes), that serve as the “readout” in a seqFISH run (FIG. 1C-D; Table 1). In principle, this multiplexing approach can be applied to studying combinations of different species (FIG. 1C) or for pooling bacteria from different growth conditions (FIG. 1D). We validated the latter application by individually labeling the 16S rRNAs of each of the 11 growth curve samples with unique Ribo-Tags. The samples were pooled, collectively hybridized with the 105 gene probe library, and subjected to sequential hybridizations to measure gene-expression and to decode cell identity (FIG. 2B). We acquired expression profiles for >50,000 individual P. aeruginosa cells, over 91.8% of which were unambiguously decoded and assigned to the condition from which they originated (FIG. 2B). We estimate the false positive decoding rate at 0.04% (1 in 2500 cells) by counting the number of hits for barcodes left out of the experiment, demonstrating both high efficiency and accuracy for par-seqFISH.

In addition to acquiring mRNA expression profiles, our imaging-based platform permits concurrent tracking of key information such as cell size and shape, and can be combined with functional stains, markers and/or immunofluorescence measurements (Takei et al., 2021). This opens up the possibility of correlating particular expression profiles at the single cell level with integrative physiological or cell biological parameters. We applied a 4′,6-diamidino-2-phenylindole (DAPI) stain as a part of the par-seqFISH experiment and used DAPI fluorescence to estimate the nucleoid size and chromosome copy per cell. Comparing cells at different stages of growth shows that both nucleoid size (estimating cell size) and chromosome number distributions follow identical trends, in agreement with the P. aeruginosa literature (Vallet-Gely and Boccard, 2013) (FIG. 2C-D). We also estimated ribosome abundance using 16S rRNA fluorescence. Notably, this metric differed significantly from the chromosome parameters, displaying contrasting intensities at different stages of lag phase, increased variability at deep stationary and a delay in signal decline during the shift from exponential growth to stationary phase (FIG. 2E). In contrast, the total number of mRNAs per cell (estimated by our 105 genes) differentiates each time point along the growth curve, reaching a maxima and minima at the fastest and slowest growth rates, respectively (FIG. 2F). These data further support the accuracy of our par-seqFISH multiplexing approach and demonstrate the unique ability of this method to integrate single cell gene-expression with global parameters.

To examine whether our expression profiles faithfully capture known physiological processes that occur during culture development, we grouped the cells according to their decoded conditions and calculated their average gene expression profiles. We find a temporally resolved expression pattern associated with different stages of growth (FIG. 2G). For example, genes representing high replicative/biosynthetic capacity such as those involved in RNA and protein biosynthesis reach their peak expression during maximal division rate but decreased between 90 to 250-fold in stationary phase (FIG. 2G). In contrast, stress factors involved in stationary phase adaptation and nutrient limitation peak at low division rates and higher cell densities (FIG. 2G). QS signal production, receptor expression and target activation reflect the known hierarchical QS regulatory network (Lee and Zhang, 2015). Notably, the expression of anaerobic metabolism genes occurred in two stages: early induction of the fermentation and nitrate/nitrite reduction genes in the entry to stationary phase, in which hypoxic conditions emerge, followed by expression of the remaining denitrification pathway at lower predicted oxygen levels (Price-Whelan et al., 2007) (FIG. 2G). Furthermore, the shift from aerobic to anaerobic metabolism was accompanied by sequential exchanges in terminal oxidase identities, from ccoN1 to ccoN2 and finally, ccoN4, concomitantly with the induction of phenazine biosynthesis (Arai, 2011; Jo et al., 2017) (FIG. 2G).

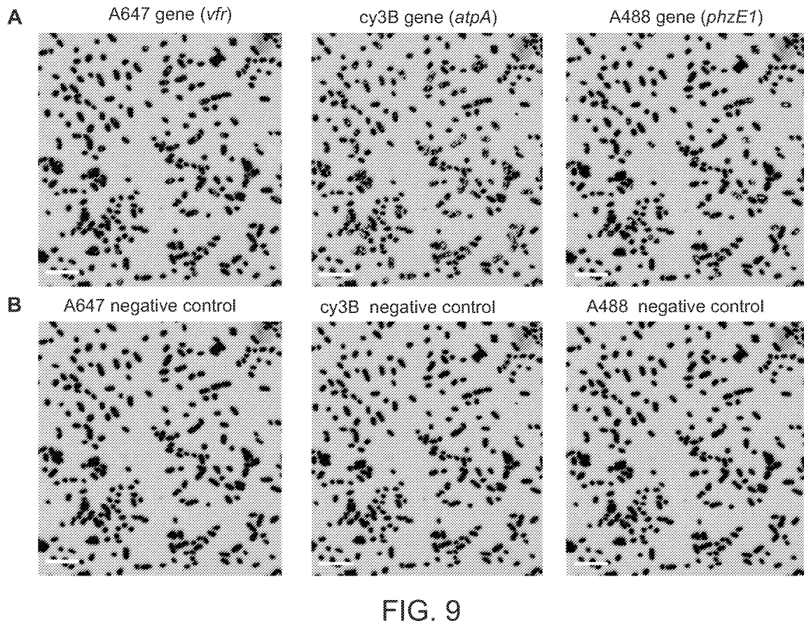

Notably, repeated mRNA measurements of the same genes in independent and spaced hybridization rounds were well correlated, both in average expression and at single-bacterium levels (Pearson R=0.86, 0.89 and 0.9, for sigX, rpsC and rpoS, respectively). In addition, the three negative control genes had an average false positive rate of 0.002 transcripts per cell (FIG. 9). Together, these results further validate the accuracy of our multiplexing method and demonstrate that our marker genes capture diverse transcriptional states across a wide range of physiological conditions.

### Experiment 2

### Transient Emergence of Physiologically Distinct Sub-Populations During Lb Growth

Phenotypic diversity in clonal populations can generate distinct sub-populations that specialize in different tasks at different times, setting a fertile ground for bet-hedging behaviors and complex interactions (Ackermann, 2015; Rosenthal et al., 2018). The single-cell resolution and high sensitivity of seqFISH has the potential to shed light on this important yet largely unexplored aspect of microbial life.

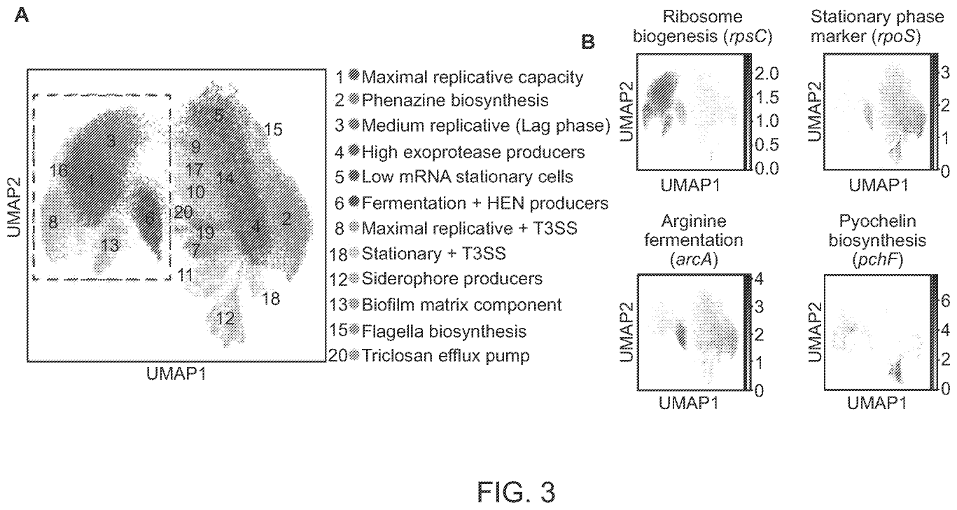

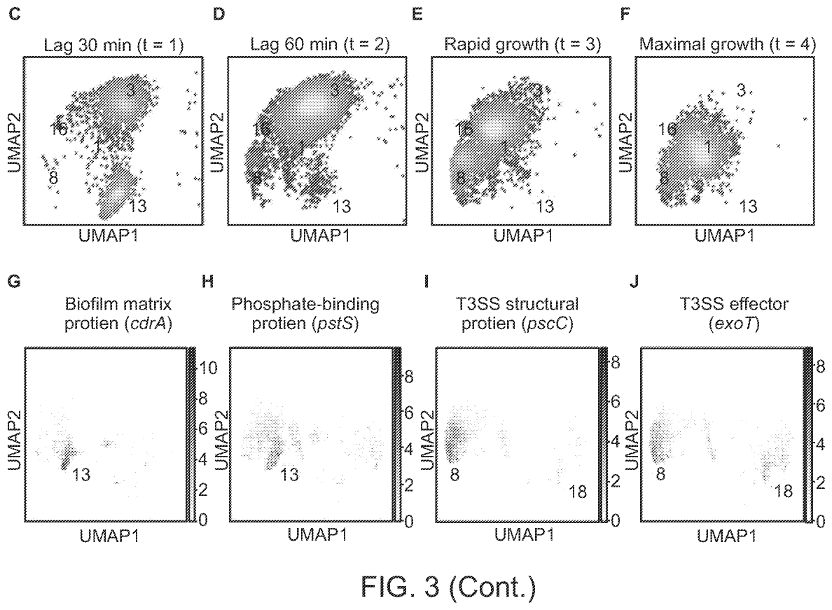

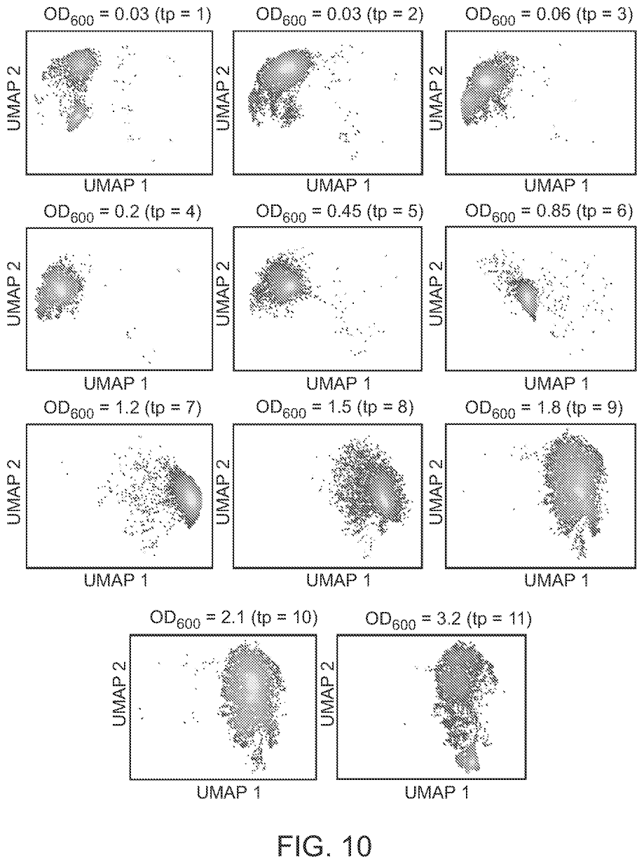

We applied Uniform Manifold Approximation and Projection (UMAP) dimensionality reduction and unsupervised clustering to our single-cell expression data (McInnes et al., 2018). This analysis charted the single-cell phenotypic landscape in LB growth, from the perspective of our marker genes. Analyzing the 11 time points together, we detect 20 sub-populations with diverse predicted functional capabilities. These included among others, differential replicative capacity, exoproduct biosynthesis, and virulence factor production (FIG. 3A-B). We find that the sampled populations of most of the growth conditions are partitioned into multiple co-existing sub-groups with distinct expression profiles (FIG. 3; FIG. 10; See Table S4: U.S. Provisional Patent Application No. 63/153,234, filed Feb. 24, 2021). Notably, our data suggest that the degree of dispersion within this expression space (estimating phenotypic diversity) varies significantly between conditions and is elevated during stationary phase (FIG. 10; See Table S4: U.S. Provisional Patent Application No. 63/153,234, filed Feb. 24, 2021).

Our growth condition-specific analysis revealed intriguing dynamics during lag phase progression. It could be expected that lag phase cultures will follow a steady ribosome accumulation as the cells progress toward exponential growth and maximal ribosome content (Bosdriesz et al., 2015). In contrast, we found a relative decline in the average ribosomal rRNA levels: early lag phase populations (30 min post dilution) had a higher signal than the more advanced lag culture (60 min post dilution; FIG. 2E). These differences appear to be rooted in the transient emergence and disappearance of an early lag sub-population with exceptionally high levels of 16S rRNA (cluster 13; comprising 34.6% of the population in early lag; FIG. 3C-F; FIG. 11; See U.S. Provisional Patent Application No. 63/153,234, filed Feb. 24, 2021Table S4). In agreement with the deviation in the rRNA signal, this sub-population also shows a proportional increase in total mRNA counts. However, its size and chromosome copy distributions were not elevated (FIG. 11; cluster 13 vs. 3).

Beyond illuminating the extent of heterogeneity in seemingly well mixed cultures and classifying subpopulations into particular types, seqFISH can directly connect global cell-specific parameters such as ribosome levels or cell shape to particular gene-expression signatures. For example, a closer examination of the metabolically hyperactive sub-population revealed a 186-fold enrichment in cdrA expression relative to the rest of the population (FIG. 3G). The cdrA gene encodes a major adhesive protein component of the P. aeruginosa biofilm matrix (Borlee et al., 2010; Reichhardt et al., 2018). Expression of cdrA is commonly used as a reporter for cyclic diguanylate monophosphate (c-di-GMP) levels, a key signaling molecule involved in surface attachment (Armbruster et al., 2019). In addition, this sub-population also displays a 30-fold enrichment in pstS expression, which encodes for the phosphate-binding component of the pstSCAB phosphate uptake system (FIG. 3H). PstS has been previously detected in extracellular appendages of P. aeruginosa and has been suggested to provide an adhesion phenotype to intestinal epithelial cells (Zaborina et al., 2008). In support of this non-canonical role, pstS was recently suggested to confer a similar adherence phenotype in Acinetobacter baumannii, another human pathogenic bacterium (Gil-Marques et al., 2020).

A second example from our dataset of the type of fine-grained information seqFISH can provide comes from the temporal expression of genes involved in virulence factor production. Single cell variation in virulence factor production has been suggested as a mechanism for division of labor during infection (Diard et al., 2013). P. aeruginosa employs a variety of virulence factors to overcome the host immune response (Malhotra et al., 2019), including the type 3 secretion system (T3SS) that translocate toxins (effectors) directly into host cells (Hauser, 2009). Our gene set monitors two T3SS structural genes (pscC and perD) and two main effectors (exoT and exoY), all of which are encoded in different operons (Wurtzel et al., 2012). We detected two different types of sub-populations with enriched T3SS related genes, suggesting a unique division of cells into virulent and avirulent states (FIG. 3I-J). The first group transiently appears during exponential growth and constitutes 8-30% of the population (FIGS. 3C-F and 3I-J; See Table S4: U.S. Provisional Patent Application No. 63/153,234, filed Feb. 24, 2021). This group expresses both the secretion system genes (86-fold enrichment) and the effectors (28-fold). In contrast, the second group appears 3-4 divisions later, close to the replicative minima at stationary phase, and occupies only ˜2.7% of cells (See Table S4: U.S. Provisional Patent Application No. 63/153,234, filed Feb. 24, 2021). This sub-population is strongly enriched for the two effectors (average 26-fold; FIG. 3I-J) but only mildly so for the secretion system (6-fold), as compared with the earlier group.

We can potentially reconcile these observations as follows: P. aeruginosa has been shown to contain approximately 1-3 T3SS units per cell under inducing conditions (Lombardi et al., 2019). Thus, successive divisions following T3SS expression will result in dilution of the T3SS⁺ group. Assuming the inheritance of the T3SS and effectors is uncoupled, then T3SS⁺ stationary phase cells are likely to lose their effectors during division and are predicted to be “inactive”. Thus, an intriguing hypothesis is that P. aeruginosa invests in the costly T3SS⁺ sub-population during “times of plenty” (rapid growth) and specifically expresses the effectors at stationary to “reload” and maintain this sub-population following division-based dilution, just prior to growth arrest. Together, these examples underscore the power of seqFISH to suggest hypotheses that can be tested going forward.

### Experiment 3

### Spatial Transcriptomics at a Single-Cell Resolution in P. Aeruginosa Biofilms

Though much can be learned by applying seqFISH to planktonic cultures, in many contexts, bacteria exist in biofilms (Flemming et al., 2016). Variation in local environmental conditions and the effect of spatially confined metabolic activities in biofilm populations can promote the emergence of chemically distinct microenvironments and phenotypes (Evans et al., 2020). We reasoned that seqFISH's capacity to record transcriptional activities with micron resolution would be particularly useful in shedding light on these processes.

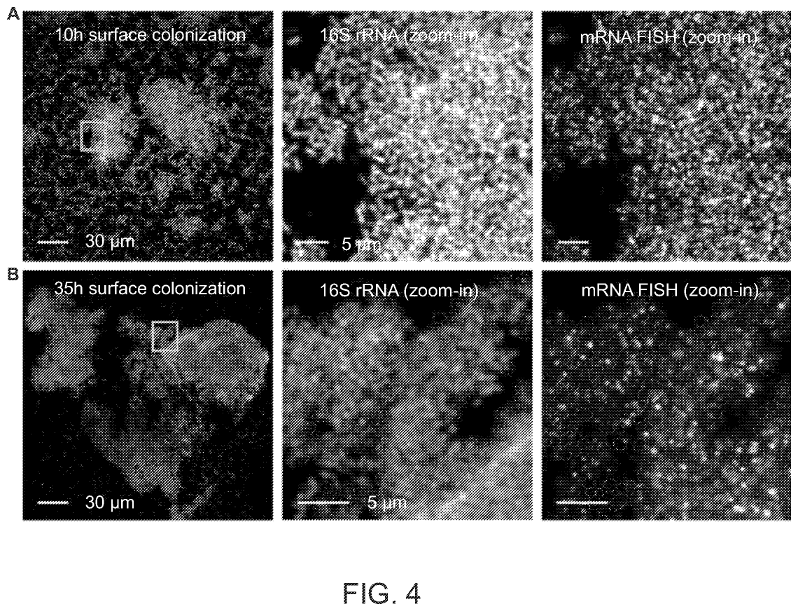

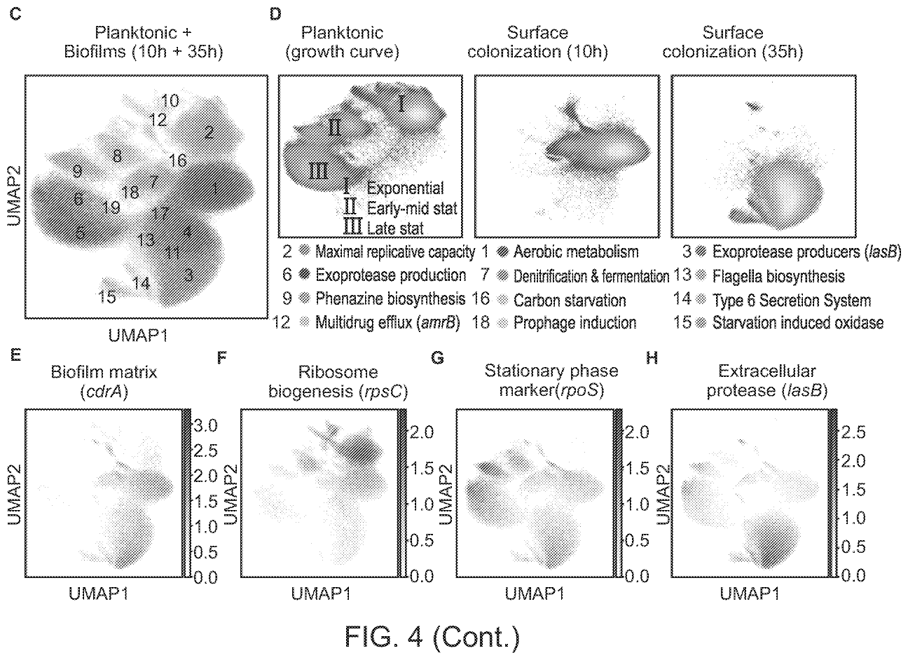

The P. aeruginosa biofilm mode of life is particularly important in chronic infections such as those residing in the airways of individuals with CF (Bjarnsholt et al., 2009; Hoiby et al., 2011). Accordingly, having used LB to validate bacterial seqFISH, we switched to synthetic cystic fibrosis sputum medium (SCFM) for our biofilm studies (Palmer et al., 2007). Briefly, bacteria were incubated in coverslip attached microwells and the medium was replaced every several hours (Methods). Using biofilms that were allowed to develop for 10 or 35 hours, we imaged hundreds of aggregates ranging in size from several bacteria to tens-of-thousands of tightly bound members (FIG. 4A-B). As a reference for cellular physiological states, we also performed a planktonic growth curve experiment in SCFM. We applied par-seqFISH multiplexing to image 10 time points matching those sampled in the planktonic LB experiment (Methods). We extracted the physical coordinates of individual bacterial cells within microaggregates, acquiring a microscale spatial expression profile for 365,000 surface attached bacteria (FIG. 4A-B). In addition, we collected single-cell expression data for 218,000 planktonic cells.

A basic question we sought to answer was the extent to which transcriptional responses are unique to the biofilm lifestyle. We performed a joint UMAP analysis using both biofilm and planktonic samples (FIG. 4C). These different modes of growth cluster into independent groups in expression space, reflecting their significant physiological differences (FIG. 4D). Ribosome and RNAP subunit expression in the planktonic experiment correlated strongly with growth rate, as observed in LB (FIG. 4D). Examining these marker genes in the biofilm-derived cells places the average replicative capacity of the 10 h and 35 h biofilm populations at roughly equal to those of early-mid and late stationary planktonic populations, respectively (FIG. 4F). Expression of the stationary phase master regulator, rpoS further supports this classification (FIG. 4G). However, biofilm cells also have unique expression profiles that distinguish them from liquid cultures. For example, the matrix component gene, cdrA, is uniformly expressed in both 10 and 35 h biofilms but repressed in most planktonic cells (FIG. 4E). In addition, compared with stationary liquid cells, our data indicates that early biofilms (10 h) have higher expression of sigX (5.1-fold), a transcription factor recently implicated in biofilm formation (Gicquel et al., 2013), mexB (>4.5-fold), of the mexA-mexB-oprM antibiotic efflux system, and an increase in the 3′-5′ exonuclease, polynucleotide phosphorylase (pnp) (7.5-fold). Comparing the 35 h biofilm to the stationary cells, we find a 3.3-fold increase in the extracellular protease, lasB, but reduced expression of other proteases such lasA (3-fold lower), as well as aprA and the rhamnolipid biosynthesis gene, rhlA (˜10-fold lower). Notably, these genes are quorum-sensing (QS) regulated and our liquid cultures expressed both lasA and rhlA at later time points than lasB, suggesting these differences may reflect the age of the biofilm rather than features that define the biofilm state per se.

### Experiment 4

### In Situ Analysis of Biofilm Specific Functions

The above data demonstrate that seqFISH can capture both cell states and their physical position directly within intact biofilms, providing an opportunity to examine known and new processes that contribute to biofilm development from a quantitative and highly spatially resolved perspective. To illustrate this, we focused on the expression patterns of representative genes known to define critical stages in biofilm development such as attachment, maturation and exclusion of competitors.

Motility systems such as the flagella and the type 4 pilus (T4P) are major determinant of surface colonization subsequent biofilm formation (Belas, 2014; Burrows, 2012; O'Toole and Kolter, 1998). Recent work identified an asymmetric division process coined “Touch-Seed-and-Go”, in which flagellated mother cells first attach to a surface and then produce un-flagellated daughter cells that contain the T4P. This c-di-GMP dependent phenotypic diversification enables the mother “spreader” cell to spawn multiple adherent “seed” populations (Laventie et al., 2019). This is thought to be mainly regulated by surface sensing (Laventie et al., 2019). However, how such motility-based division of labor affects the organization of biofilms at stages beyond surface attachment remains unknown.

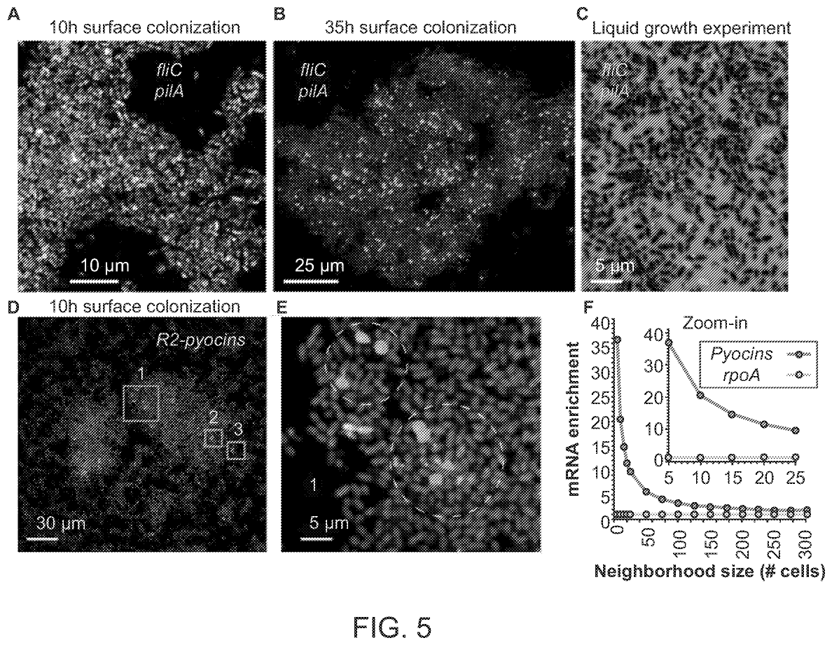

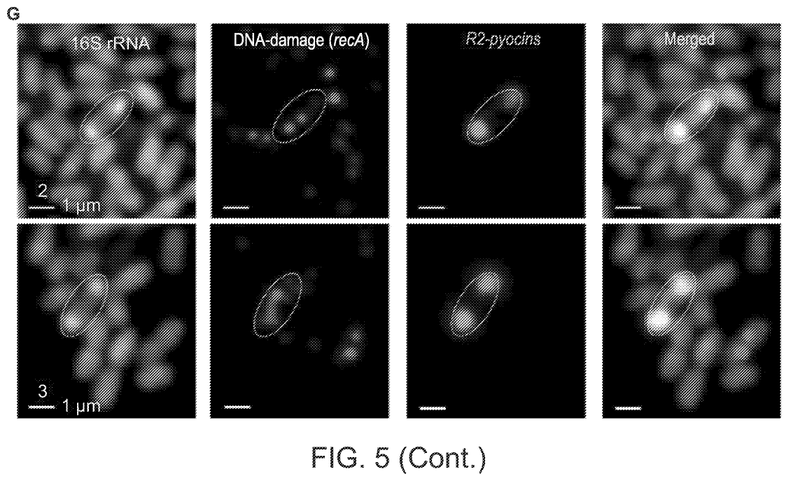

We examined the spatial expression patterns of the major flagellum and T4P components, fliC and pilA, respectively in the early surface colonization experiment (10 h biofilm). An abundant “checkerboard” like pattern is evident, in which cells express high levels of either fliC or pilA but generally not both (FIG. 5A). This pattern is apparent in both small groups (tens of cells) and in microaggregates that contain thousands of cells. In contrast, the older 35 h biofilms showed lower expression of pilA but contained a sparse but uniform distribution of fliC cells, suggesting that biofilm associated bacteria invest in a costly motility apparatus despite being spatially confined (FIG. 5B), effectively, the bacterial equivalent of purchasing a sports car during a midlife crisis. Strikingly, examining the expression of fliC and pilA in our paired planktonic experiment we find a similar mutually exclusive pattern (FIG. 5C). Thus, in contrast to the current model, our planktonic control experiment suggests that the asymmetric distribution of motility systems is unlikely to be directly regulated by surface sensing (FIG. 5C); such a conclusion would not be possible without the means to compare transcriptional activities at the single cell level.

Beyond initial surface attachment, bacteria must establish a strong foothold for colony development as well as outcompete resident microbes. One strategy that potentially address both needs is the utilization of phage tail-like bacteriocins, broadly called tailocins (Ghequire and De Mot, 2015). These elements are thought to be adapted from prophages and are applied as narrow-spectrum toxins for kin exclusion (Bobay et al., 2014; Ghequire and De Mot, 2015). However, in contrast with antibiotics, these phage taillike structures are released into the environment via explosive lysis events that kill the producer and spray the toxin locally to inhibit nearby competitors (Turnbull et al., 2016; Vacheron et al., 2021). This event also releases extracellular DNA that integrates into the biofilm matrix, structurally supporting biofilm maturation (Turnbull et al., 2016; Whitchurch et al., 2002). Yet how this “sacrificial” process is regulated within developing biofilms is not well understood.

Our UMAP analysis identified a sub-population (cluster 18; FIG. 4C) exhibiting >1000-fold enrichment in expression of the R2-pyocin operon (Pseudomonas tailocin), represented by the PA14_08150 gene. This UMAP cluster was enriched ˜4-fold in 10 h biofilm derived cells, suggesting pyocin induction is upregulated in surface attached cells. Furthermore, we find an 11-fold higher expression of the DNA-repair gene, recA, in agreement with its role in inducing pyocin expression (Brans and Hancock, 2005). Visualizing the expression of the pyocin producers, we find that induction events are spread across various microaggregates regions but often appear in local clusters (FIG. 5D-E). Indeed, we find a ˜37-fold average spatial enrichment in pyocin expression in the immediate vicinity of strong induction sites as compared with the general population (FIG. 5F). This enrichment decayed rapidly as a function of neighborhood size, suggesting a highly localized effect (FIG. 5F).

Remarkably, in addition to reporting gene-expression levels, seqFISH also reports the physical position of measured mRNA molecules at a sub-micron resolution. During this analysis we noticed that R2-pyocin transcript fluorescence generally appeared as two spots. Upon closer examination, we discovered that this mRNA is strongly localized to the two cell poles (FIG. 5G). The 16S rRNA fluorescent signal in these pyocin producers show identical polarization, a rare pattern not observed in neighboring non-inducing cells (FIG. 5G). These data suggest that ribosomes and the R2-pyocin transcript are mobilized following induction and spatially co-localize. In contrast, the expression of recA did not follow this pattern, suggesting a pyocin-specific effect (FIG. 5G). Notably, a recent study discovered an identical polar localization for two different Pseudomonas protegees R-tailocins at the protein level (Vacheron et al., 2021). Together, these data hint at a potentially conserved RNA-dependent mechanism for R-tailocin protein polar localization. We hypothesize that the spatially correlated ribosomal enrichment may provide efficient local translation and particle accumulation prior to cell lysis.

### Experiment 5

### Temporal Evolution of Metabolic Heterogeneity During Biofilm Development

Beyond resolving transcriptional activities that contribute to biofilm developmental processes, seqFISH can reveal how biofilm cells metabolically respond to subtle changes in their local microenvironment. Chemical heterogeneity is a key feature of spatially structured environments, and metabolic heterogeneity characterizes mature biofilms (Evans et al., 2020; Povolotsky et al., 2021; Schiessl et al., 2019; Wessel et al., 2014). Yet until now, it has been impossible to capture the development of fine-grained metabolic structure across multiple suites of genes at different times.

To map biofilm metabolic development, we focused on genes whose regulation and functions are well understood. In particular, we focused on catabolic genes whose gene products enable energy conservation under different oxygen concentrations. Oxygen is a central and dynamic factor that influences metabolic activity in bacterial biofilms (Dietrich et al., 2013; Evans et al., 2020; Stewart, 2003; Wessel et al., 2014). Local oxygen availability can vary significantly within structured environments and is biotically shaped within biofilms (Cowley et al., 2015; Stewart and Franklin, 2008; Wessel et al., 2014). P. aeruginosa can survive under anaerobic conditions by fermenting different substrates and/or denitrifying (Arai, 2011; Eschbach et al., 2004; Yoon et al., 2002). Accordingly, monitoring the expression of these catabolic genes as well as others that are co-regulated with them provides a means to track local oxygen availability and its dynamic effects on biofilm metabolic coordination.

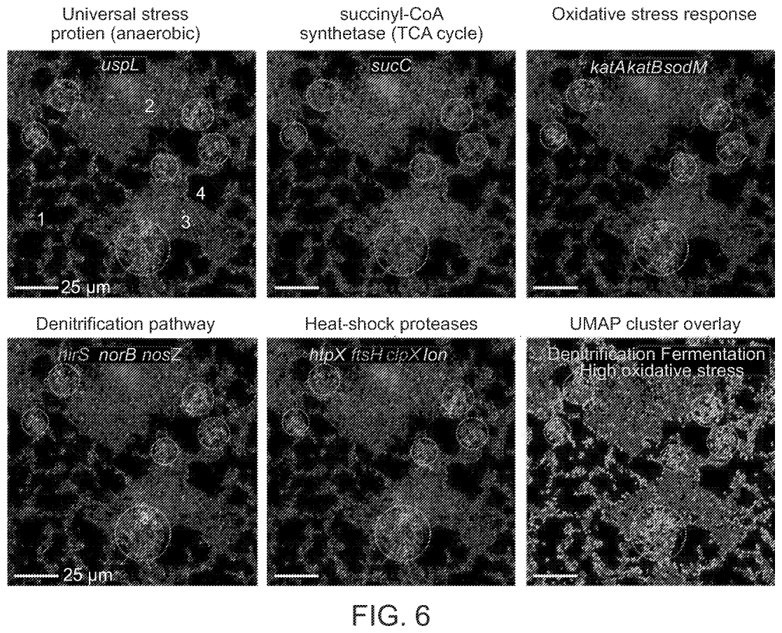

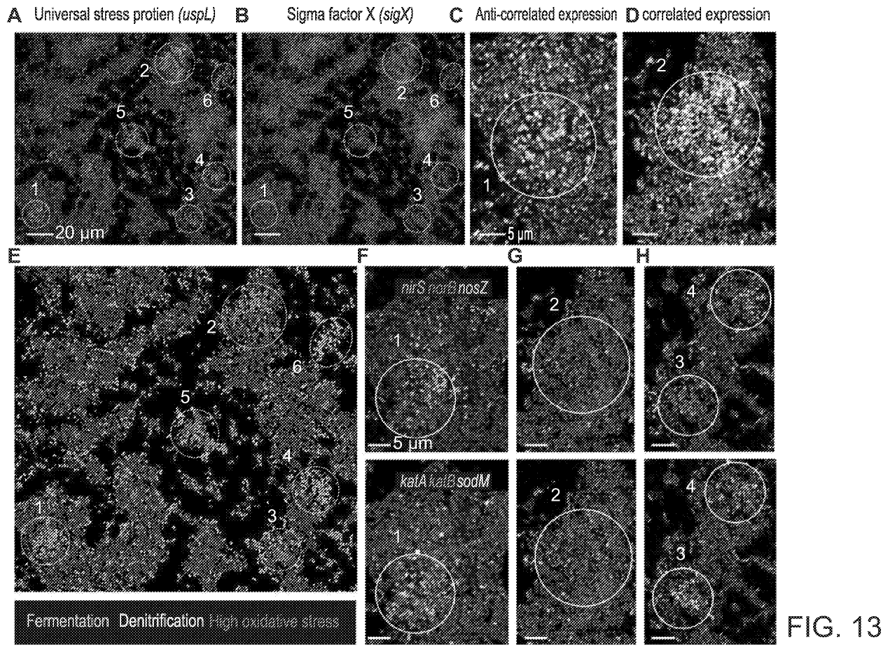

How quickly and over what spatial scales do biofilm cells metabolically differentiate? Following the uspL gene, which was strongly induced during hypoxic conditions and correlated with anaerobic fermentation and denitrification genes in our planktonic growth experiments, we observed surprisingly heterogeneous responses to oxygen depletion over just a few microns in young (10 h) biofilms (FIG. 6A). Notably, uspL expression is strongly spatially correlated with multiple anaerobic markers (FIG. 12), indicating that this gene reports on local anaerobic activities. A closer examination of these putative hypoxic sites showed a frequent anti-correlation of uspL with multiple genes that are otherwise uniformly expressed in 10 h biofilms, appearing as co-localized but reversed expression patches (FIG. 6B). Among the anti-correlated functions are the TCA cycle gene, sucC, and replicative capacity genes such as RNAP and ribosome subunits (FIG. 6B; FIGS. 12-13). However, exceptions to this anti-correlation were also visible (FIG. 13).

Can the metabolic heterogeneity revealed by oxygen-responsive marker genes provide an entry point for the discovery of more nuanced cellular responses at the microscale? Our spatial correlation analysis revealed an intriguing association between anaerobic metabolism genes, such as the denitrification pathway (narG-nirS-norB-nosZ), and the oxidative stress response genes katA, katB and sodM, encoding for the inducible catalases and an Mn-dependent superoxide dismutase, respectively (Brown et al., 1995; Hassett et al., 1992; Su et al., 2014) (FIG. 6C-D; FIGS. 12-13). Nitrite respiring P. aeruginosa produce the highly toxic intermediate nitric oxide (NO) (Cutruzzold and Frankenberg-Dinkel, 2016). Indeed, KatA was recently demonstrated to play a role in protection from NO-associated stress (Su et al., 2014), suggesting that these sub-aggregate regions correspond to microenvironments with high NO levels. In agreement with this hypothesis, we find that the stress response pattern is also spatially correlated with heat-shock protease expression, including the membrane protease, fisH, which was found to play an important role in survival under anoxic conditions (Basta et al., 2017) (FIG. 6E; FIG. 12). These data highlight how contrasting physiological states can be established just a few microns away early in biofilm development.

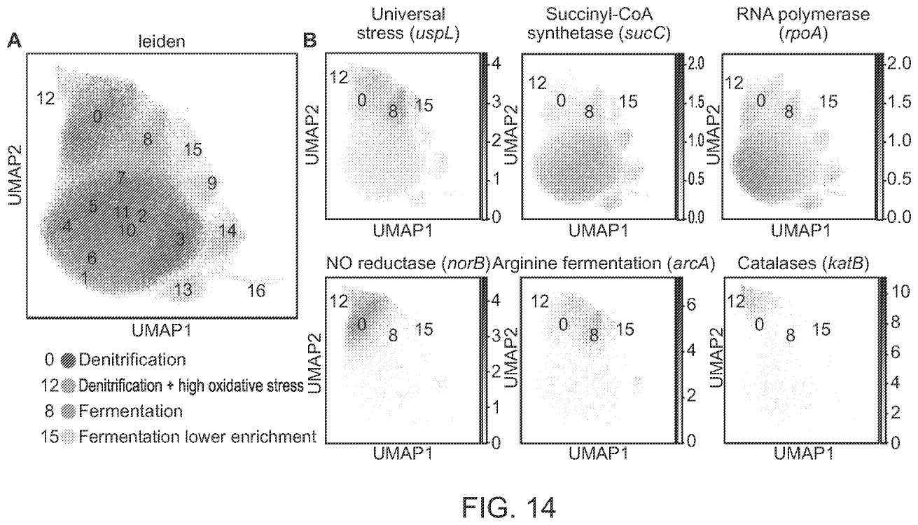

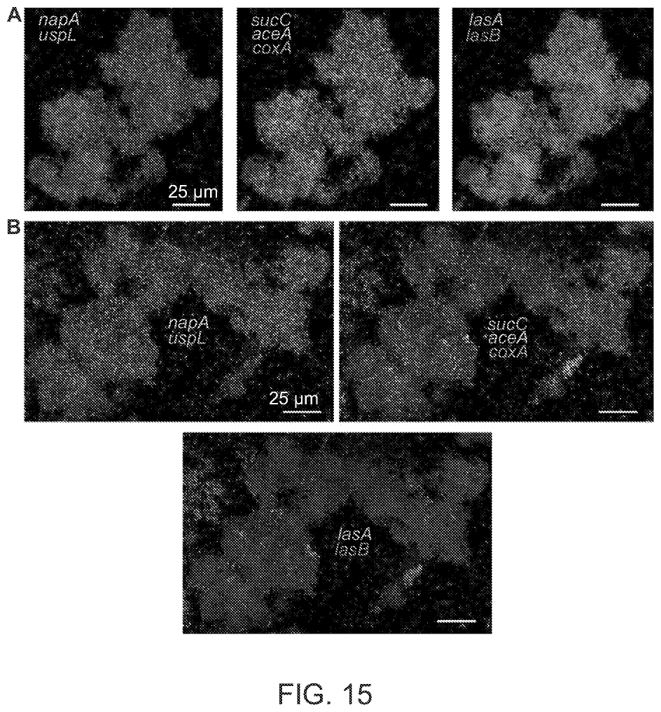

These coordinated expressions patterns for particular genes led us to hypothesize that these patterns reflected the spatiometabolic distribution of distinct physiological “states” across the biofilm. To test this hypothesis we conducted a targeted UMAP analysis using only the 10 h biofilm cells (FIG. 14). We identified two main anaerobic sub-populations corresponding to denitrification and fermentation dominated metabolic states (FIG. 14). In addition, we detected a smaller sub-population of denitrifying cells with 5.3-fold average increase in the oxidative stress factors katB, sodM, and ahpF, which encodes for an alkyl hydroperoxide reductase (Ochsner et al., 2000). Relative to the main denitrifying sub-group, stressed cells have lower expression of the denitrification pathway (˜4-fold) and a >2-fold reduction in replicative capacity marker levels (rpoA, rpsC and atpA), in support of a potentially damaged state. Projecting these single-cell metabolic states over their respective biofilm positions showed a strong overlap with the above predicted hypoxic pockets, supporting our hypothesis and revealing that multiple metabolic states can co-exist in the same patch (FIG. 6F; FIG. 15).

Given the extent of transcriptional heterogeneity manifest in young biofilms, we wondered whether such heterogeneity would persist as biofilms aged. We speculated that the higher cell densities and more committed spatial structuring of mature biofilms might favor larger scale metabolic zonation. We therefore examined the spatial expression patterns in a 35 h biofilm experiment.

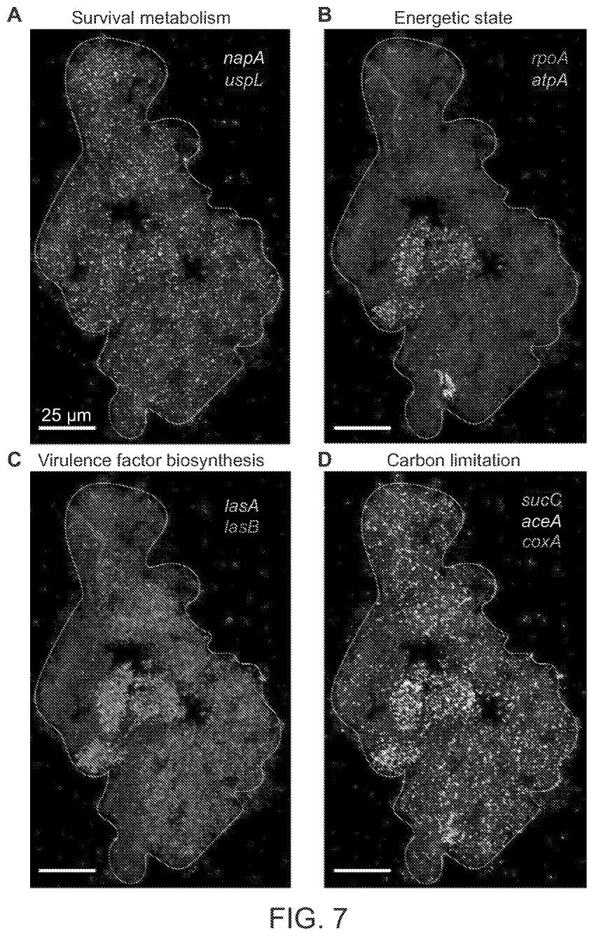

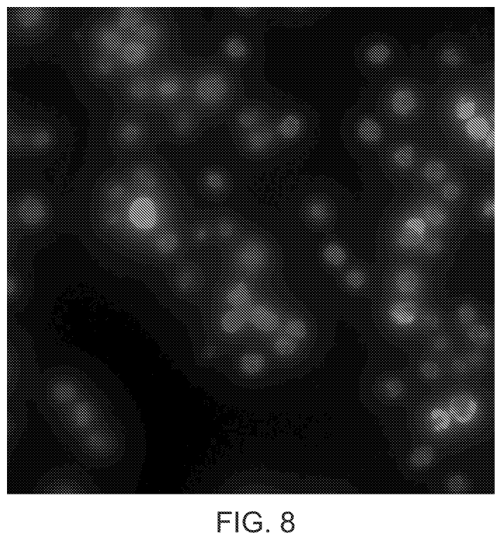

In contrast to the spatial variation in aerobic and anaerobic metabolic processes seen in 10 h biofilms, 35 h biofilms have ˜50-fold lower average expression of the denitrification pathway genes nar-nirs-norB-nosZ. Indeed, these genes are known to be repressed by the las and rhl QS-systems, indicating P. aeruginosa is programmed to shut down denitrification at high cell densities (Toyofuku et al., 2007; Yoon et al., 2002). However, in addition to this complete and co-regulated pathway, P. aeruginosa also encodes an independent periplasmic nitrate reductase (nap) (Van Alst et al., 2009; Lin et al., 2018). Intriguingly, the napA gene is uniformly expressed at low levels across the 35 h aggregates, a pattern that was closely shared with the uspL gene (FIG. 7A; FIG. 15). NapA has been implicated in maintaining redox homeostasis under oxygen limitation (Dietrich et al., 2013) and the uspL paralogue, uspK, was shown to play a role in survival under such conditions (Basta et al., 2017; Schreiber et al., 2006). At first blush, these results suggest that as aggregate cell mass grows, survival physiology dominates over growth-promoting processes on average. Yet we also find substantial and large-scale heterogeneity in certain genes, such as the replicative capacity markers (FIG. 7B; FIG. 15) and, lasB, encoding a QS-regulated extracellular protease (FIG. 7C; Figure S7). These data demonstrate that while older biofilms generally comprise larger zones of particular activities than younger biofilms, a single microaggregate can still contain cell types with distinct physiological states and virulence-related activities.

Finally, that metabolism dynamically shapes the microenvironment leads to the prediction that differences in local nutrient availability will be reflected in heterogenous transcriptional activities over small spatial scales (Evans et al., 2020). We see evidence of this phenomenon in our data when focusing on carbon metabolism, for example. Where replicative capacity appears to be high and carbon is presumably replete, we see co-expression of the TCA cycle gene (sucC) (FIG. 7B). However, when carbon is limiting, bacteria can utilize the glyoxylate shunt (GS), which bypasses the oxidative decarboxylation steps of the TCA. The GS provides an alternative metabolic pathway for utilizing acetate and fatty acids as carbon sources (Crousilles et al., 2018; Dolan and Welch, 2018). In the GS, carbon flux is redirected by isocitrate lyase (ICL) which competes with the TCA enzyme isocitrate dehydrogenase (ICD) for isocitrate. However, since ICD has a much lower Km it must be enzymatically inactivated via phosphorylation for the carbon flux to be redirected to the GS (Crousilles et al., 2018). However, little is still known about the transcriptional regulation of these pathways (Dolan et al., 2020). Our gene set contains both the GS gene, aceA, as well as a downstream TCA cycle gene, sucC. While these genes are often co-expressed, we find that only the GS marker, aceA, is expressed in low energetic capacity biofilm zones (FIG. 7D; FIG. 15), suggesting these subregions experience carbon limitation. In support of this hypothesis, these regions also express the tightly regulated terminal oxidase gene, coxA, which is transcriptionally induced by carbon starvation, a condition in which it promotes survival (Basta et al., 2017; Kawakami et al., 2010) (FIG. 7D; FIG. 15). This is just one example of the type of coherent spatiometabolic stratification pattern seqFISH can reveal at a given moment in time.

### REFERENCES

The following references are incorporate by their entirety.
Ackermann, M. (2015). A functional perspective on phenotypic heterogeneity in microorganisms. Nat. Rev. Microbiol. 13, 497-508.
Van Alst, N. E., Sherrill, L. A., Iglewski, B. H., and Haidaris, C. G. (2009). Compensatory periplasmic nitrate reductase activity supports anaerobic growth of Pseudomonas aeruginosa PA01 in the absence of membrane nitrate reductase. Can. J. Microbiol. 55, 1133-1144.
Arai, H. (2011). Regulation and Function of Versatile Aerobic and Anaerobic Respiratory Metabolism in Pseudomonas aeruginosa. Front. Microbiol. 2, 103.
Armbruster, C. R., Lee, C. K., Parker-Gilham, J., de Anda, J., Xia, A., Zhao, K., Murakami, K., Tseng, B. S., Hoffman, L. R., Jin, F., et al. (2019). Heterogeneity in surface sensing suggests a division of labor in Pseudomonas aeruginosa populations. Elife 8.
Babin, B. M., Atangcho, L., van Eldijk, M. B., Sweredoski, M. J., Moradian, A., Hess, S., Tolker-Nielsen, T., Newman, D. K., and Tirrell, D. A. (2017). Selective Proteomic Analysis of Antibiotic-Tolerant Cellular Subpopulations in Pseudomonas aeruginosa Biofilms. MBio 8.
Basta, D. W., Bergkessel, M., and Newman, D. K. (2017). Identification of fitness determinants during energy-limited growth arrest in Pseudomonas aeruginosa. MBio 8, e01170-17.
Belas, R. (2014). Biofilms, flagella, and mechanosensing of surfaces by bacteria. Trends Microbiol. 22, 517-527.
Berg, S., Kutra, D., Kroeger, T., Straehle, C. N., Kausler, B. X., Haubold, C., Schiegg, M., Ales, J., Beier, T., Rudy, M., et al. (2019). ilastik: interactive machine learning for (bio)image analysis. Nat. Methods 16, 1226-1232.
Bhagirath, A. Y., Li, Y., Somayajula, D., Dadashi, M., Badr, S., and Duan, K. (2016). Cystic fibrosis lung environment and Pseudomonas aeruginosa infection. BMC Pulm Med 16, 174.
Bjarnsholt, T., Jensen, P. O., Fiandaca, M. J., Pedersen, J., Hansen, C. R., Andersen, C. B., Pressler, T., Givskov, M., and Hoiby, N. (2009). Pseudomonas aeruginosa biofilms in the respiratory tract of cystic fibrosis patients. Pediatr. Pulmonol. 44, 547-558.
Blattman, S. B., Jiang, W., Oikonomou, P., and Tavazoie, S. (2020). Prokaryotic single-cell RNA sequencing by in situ combinatorial indexing. Nat. Microbiol. 5, 1192-1201.
Bobay, L.-M., Touchon, M., and Rocha, E. P. C. (2014). Pervasive domestication of defective prophages by bacteria. Proc. Natl. Acad. Sci. USA 111, 12127-12132.
Bocci, F., Suzuki, Y., Lu, M., and Onuchic, J. N. (2018). Role of metabolic spatiotemporal dynamics in regulating biofilm colony expansion. Proc. Natl. Acad. Sci. USA 115, 4288-4293.
Borlee, B. R., Goldman, A. D., Murakami, K., Samudrala, R., Wozniak, D. J., and Parsek, M. R. (2010).
Pseudomonas aeruginosa uses a cyclic-di-GMP-regulated adhesin to reinforce the biofilm extracellular matrix. Mol. Microbiol. 75, 827-842.
Bosdriesz, E., Molenaar, D., Teusink, B., and Bruggeman, F. J. (2015). How fast-growing bacteria robustly tune their ribosome concentration to approximate growth-rate maximization. FEBS J. 282, 2029-2044.
Brans, M. D., and Hancock, R. E. W. (2005). Ciprofloxacin induction of a susceptibility determinant in Pseudomonas aeruginosa. Antimicrob. Agents Chemother. 49, 3222-3227.
Brown, S. M., Howell, M. L., Vasil, M. L., Anderson, A. J., and Hassett, D. J. (1995). Cloning and characterization of the katB gene of Pseudomonas aeruginosa encoding a hydrogen peroxide-inducible catalase: purification of KatB, cellular localization, and demonstration that it is essential for optimal resistance to hydrogen peroxide. J. Bacteriol. 177, 6536-6544.
Burrows, L. L. (2012). Pseudomonas aeruginosa twitching motility: type IV pili in action. Annu. Rev. Microbiol. 66, 493-520.
Chadwick, G. L., Jimenez Otero, F., Gralnick, J. A., Bond, D. R., and Orphan, V. J. (2019). NanoSIMS imaging reveals metabolic stratification within current-producing biofilms. Proc. Natl. Acad. Sci. USA 116, 20716-20724.
Chen, K. H., Boettiger, A. N., Moffitt, J. R., Wang, S., and Zhuang, X. (2015). RNA imaging. Spatially resolved, highly multiplexed RNA profiling in single cells. Science 348, aaa6090.
Choi, H. M. T., Beck, V. A., and Pierce, N. A. (2014). Next-generation in situ hybridization chain reaction: higher gain, lower cost, greater durability. ACS Nano 8, 4284-4294.
Cordero, O. X., and Datta, M. S. (2016). Microbial interactions and community assembly at microscales. Curr. Opin. Microbiol. 31, 227-234.
Cowley, E. S., Kopf, S. H., LaRiviere, A., Ziebis, W., and Newman, D. K. (2015). Pediatric cystic fibrosis sputum can be chemically dynamic, anoxic, and extremely reduced due to hydrogen sulfide formation. MBio 6, e00767.
Crousilles, A., Dolan, S. K., Brear, P., Chirgadze, D. Y., and Welch, M. (2018). Gluconeogenic precursor availability regulates flux through the glyoxylate shunt in Pseudomonas aeruginosa. J. Biol. Chem. 293, 14260-14269.
Cutruzzold, F., and Frankenberg-Dinkel, N. (2016). Origin and Impact of Nitric Oxide in Pseudomonas aeruginosa Biofilms. J. Bacteriol. 198, 55-65.
DePas, W. H., Starwalt-Lee, R., Van Sambeek, L., Ravindra Kumar, S., Gradinaru, V., and Newman, D. K. (2016). Exposing the three-dimensional biogeography and metabolic states of pathogens in cystic fibrosis sputum via hydrogel embedding, clearing, and rRNA labeling. MBio 7, e00796-16.
Diard, M., Garcia, V., Maier, L., Remus-Emsermann, M. N. P., Regoes, R. R., Ackermann, M., and Hardt, W.-D. (2013). Stabilization of cooperative virulence by the expression of an avirulent phenotype. Nature 494, 353-356.
Dietrich, L. E. P., Okegbe, C., Price-Whelan, A., Sakhtah, H., Hunter, R. C., and Newman, D. K. (2013).
Bacterial community morphogenesis is intimately linked to the intracellular redox state. J. Bacteriol. 195, 1371-1380.
Dolan, S. K., and Welch, M. (2018). The glyoxylate shunt, 60 years on. Annu. Rev. Microbiol. 72, 309-330.
Dolan, S. K., Pereira, G., Silva-Rocha, R., and Welch, M. (2020). Transcriptional regulation of central carbon metabolism in Pseudomonas aeruginosa. Microb Biotechnol 13, 285-289.
Edelstein, A., Amodaj, N., Hoover, K., Vale, R., and Stuurman, N. (2010). Computer control of microscopes using μManager. Curr. Protoc. Mol. Biol. Chapter 14, Unit14.20.
Eng, C.-H. L., Lawson, M., Zhu, Q., Dries, R., Koulena, N., Takei, Y., Yun, J., Cronin, C., Karp, C., Yuan, G.-C., et al. (2019). Transcriptome-scale super-resolved imaging in tissues by RNA seqFISH. Nature 568, 235-239.
Eschbach, M., Schreiber, K., Trunk, K., Buer, J., Jahn, D., and Schobert, M. (2004). Long-term anaerobic survival of the opportunistic pathogen Pseudomonas aeruginosa via pyruvate fermentation. J. Bacteriol. 186, 4596-4604.
Evans, C. R., Kempes, C. P., Price-Whelan, A., and Dietrich, L. E. P. (2020). Metabolic Heterogeneity and Cross-Feeding in Bacterial Multicellular Systems. Trends Microbiol.
Fei, J., and Sharma, C. M. (2018). RNA localization in bacteria. Microbiol. Spectr. 6.
Femino, A. M., Fay, F. S., Fogarty, K., and Singer, R. H. (1998). Visualization of single RNA transcripts in situ. Science 280, 585-590.
Flemming, H.-C., and Wuertz, S. (2019). Bacteria and archaea on Earth and their abundance in biofilms. Nat. Rev. Microbiol. 17, 247-260.
Flemming, H.-C., Wingender, J., Szewzyk, U., Steinberg, P., Rice, S. A., and Kjelleberg, S. (2016).
Biofilms: an emergent form of bacterial life. Nat. Rev. Microbiol. 14, 563-575.
Gasperotti, A., Brameyer, S., Fabiani, F., and Jung, K. (2019). Phenotypic heterogeneity of microbial populations under nutrient limitation. Curr. Opin. Biotechnol. 62, 160-167.
Ghequire, M. G. K., and De Mot, R. (2015). The tailocin tale: peeling off phage tails. Trends Microbiol. 23, 587-590.
Gicquel, G., Bouffartigues, E., Bains, M., Oxaran, V., Rosay, T., Lesouhaitier, O., Connil, N., Bazire, A., Maillot, O., Bénard, M., et al. (2013). The extra-cytoplasmic function sigma factor sigX modulates biofilm and virulence-related properties in Pseudomonas aeruginosa. PLoS One 8, e80407.
Gil-Marqués, M. L., Labrador Herrera, G., Miró Canturri, A., Pachon, J., Smani, Y., and Pachón-Ibáñez, M. E. (2020). Role of PstS in the Pathogenesis of Acinetobacter baumannii Under Microaerobiosis and Normoxia. J. Infect. Dis. 222, 1204-1212.
Hassett, D. J., Charniga, L., Bean, K., Ohman, D. E., and Cohen, M. S. (1992). Response of Pseudomonas aeruginosa to pyocyanin: mechanisms of resistance, antioxidant defenses, and demonstration of a manganese-cofactored superoxide dismutase. Infect. Immun. 60, 328-336.
Hatzenpichler, R., Krukenberg, V., Spietz, R. L., and Jay, Z. J. (2020). Next-generation physiology approaches to study microbiome function at single cell level. Nat. Rev. Microbiol. 18, 241-256.
Hauser, A. R. (2009). The type III secretion system of Pseudomonas aeruginosa: infection by injection. Nat. Rev. Microbiol. 7, 654-665.
Hoiby, N., Ciofu, O., Johansen, H. K., Song, Z., Moser, C., Jensen, P. O., Molin, S., Givskov, M., TolkerNielsen, T., and Bjarnsholt, T. (2011). The clinical impact of bacterial biofilms. Int. J. Oral Sci. 3, 55-65.
Imdahl, F., Vafadarnejad, E., Homberger, C., Saliba, A.-E., and Vogel, J. (2020). Single-cell RNAsequencing reports growth-condition-specific global transcriptomes of individual bacteria. Nat. Microbiol. 5, 1202-1206.
Jo, J., Cortez, K. L., Cornell, W. C., Price-Whelan, A., and Dietrich, L. E. (2017). An orphan cbb3-type cytochrome oxidase subunit supports Pseudomonas aeruginosa biofilm growth and virulence. Elife 6.
Jorth, P., Spero, M. A., Livingston, J., and Newman, D. K. (2019). Quantitative Visualization of Gene Expression in Mucoid and Nonmucoid Pseudomonas aeruginosa Aggregates Reveals Localized Peak Expression of Alginate in the Hypoxic Zone. MBio 10.
Kawakami, T., Kuroki, M., Ishii, M., Igarashi, Y., and Arai, H. (2010). Differential expression of multiple terminal oxidases for aerobic respiration in Pseudomonas aeruginosa. Environ. Microbiol. 12, 1399-1412.
Kopf, S. H., McGlynn, S. E., Green-Saxena, A., Guan, Y., Newman, D. K., and Orphan, V. J. (2015). Heavy water and (15) N labelling with NanoSIMS analysis reveals growth rate-dependent metabolic heterogeneity in chemostats. Environ. Microbiol. 17, 2542-2556.
Kotte, O., Volkmer, B., Radzikowski, J. L., and Heinemann, M. (2014). Phenotypic bistability in Escherichia coli's central carbon metabolism. Mol. Syst. Biol. 10, 736.
Kuchina, A., Brenner, L. M., Paleologu, L., Roco, C. M., Rosenberg, A. B., Carignano, A., Kibler, R., Hirano, M., DePaolo, R. W., and Seelig, G. (2021). Microbial single-cell RNA sequencing by split-pool barcoding. Science 371.
Laventie, B.-J., Sangermani, M., Estermann, F., Manfredi, P., Planes, R., Hug, I., Jaeger, T., Meunier, E., Broz, P., and Jenal, U. (2019). A Surface-Induced Asymmetric Program Promotes Tissue Colonization by Pseudomonas aeruginosa. Cell Host Microbe 25, 140-152.e6.
Lee, J., and Zhang, L. (2015). The hierarchy quorum sensing network in Pseudomonas aeruginosa. Protein Cell 6, 26-41.
Lignell, A., Kerosuo, L., Streichan, S. J., Cai, L., and Bronner, M. E. (2017). Identification of a neural crest stem cell niche by Spatial Genomic Analysis. Nat. Commun. 8, 1830.
Lin, Y.-C., Sekedat, M. D., Cornell, W. C., Silva, G. M., Okegbe, C., Price-Whelan, A., Vogel, C., and Dietrich, L. E. P. (2018). Phenazines Regulate Nap-Dependent Denitrification in Pseudomonas aeruginosa Biofilms. J. Bacteriol. 200.
Lombardi, C., Tolchard, J., Bouillot, S., Signor, L., Gebus, C., Liebl, D., Fenel, D., Teulon, J.-M., Brock, J., Habenstein, B., et al. (2019). Structural and Functional Characterization of the Type Three Secretion System (T3SS) Needle of Pseudomonas aeruginosa. Front. Microbiol. 10, 573.
Lubeck, E., Coskun, A. F., Zhiyentayev, T., Ahmad, M., and Cai, L. (2014). Single-cell in situ RNA profiling by sequential hybridization. Nat. Methods 11, 360-361.
Malhotra, S., Hayes, D., and Wozniak, D. J. (2019). Cystic Fibrosis and Pseudomonas aeruginosa: the Host-Microbe Interface. Clin. Microbiol. Rev. 32. Mark Welch, J. L., Rossetti, B. J., Rieken, C. W., Dewhirst, F. E., and Borisy, G. G. (2016). Biogeography of a human oral microbiome at the micron scale. Proc. Natl. Acad. Sci. USA 113, E791-800.
McInnes, L., Healy, J., and Melville, J. (2018). UMAP: Uniform Manifold Approximation and Projection for Dimension Reduction. arXiv.
Milo, R., Jorgensen, P., Moran, U., Weber, G., and Springer, M. (2010). BioNumbers—the database of key numbers in molecular and cell biology. Nucleic Acids Res. 38, D750-3.
Moffitt, J. R., Bambah-Mukku, D., Eichhorn, S. W., Vaughn, E., Shekhar, K., Perez, J. D., Rubinstein, N. D., Hao, J., Regev, A., Dulac, C., et al. (2018). Molecular, spatial, and functional single-cell profiling of the hypothalamic preoptic region. Science 362.
Nadell, C. D., Drescher, K., and Foster, K. R. (2016). Spatial structure, cooperation and competition in biofilms. Nat. Rev. Microbiol. 14, 589-600.
O'Toole, G. A., and Kolter, R. (1998). Flagellar and twitching motility are necessary for Pseudomonas aeruginosa biofilm development. Mol. Microbiol. 30, 295-304.
Ochsner, U. A., Vasil, M. L., Alsabbagh, E., Parvatiyar, K., and Hassett, D. J. (2000). Role of the Pseudomonas aeruginosa oxyR-recG operon in oxidative stress defense and DNA repair: OxyRdependent regulation of katB-ankB, ahpB, and ahpC-ahpF. J. Bacteriol. 182, 4533-4544.
Palmer, K. L., Aye, L. M., and Whiteley, M. (2007). Nutritional cues control Pseudomonas aeruginosa multicellular behavior in cystic fibrosis sputum. J. Bacteriol. 189, 8079-8087.
Povolotsky, T. L., Keren-Paz, A., and Kolodkin-Gal, I. (2021). Metabolic microenvironments drive microbial differentiation and antibiotic resistance. Trends Genet. 37, 4-8.
Price-Whelan, A., Dietrich, L. E. P., and Newman, D. K. (2007). Pyocyanin alters redox homeostasis and carbon flux through central metabolic pathways in Pseudomonas aeruginosa PA14. J. Bacteriol. 189, 6372-6381.
Raj, A., van den Bogaard, P., Rifkin, S. A., van Oudenaarden, A., and Tyagi, S. (2008). Imaging individual mRNA molecules using multiple singly labeled probes. Nat. Methods 5, 877-879.
Reichhardt, C., Wong, C., Passos da Silva, D., Wozniak, D. J., and Parsek, M. R. (2018). CdrA Interactions within the Pseudomonas aeruginosa Biofilm Matrix Safeguard It from Proteolysis and Promote Cellular Packing. MBio 9:01376-18.
Rosenthal, A. Z., Qi, Y., Hormoz, S., Park, J., Li, S. H.-J., and Elowitz, M. B. (2018). Metabolic interactions between dynamic bacterial subpopulations. Elife 7, e33099.
Schaber, J. A., Triffo, W. J., Suh, S. J., Oliver, J. W., Hastert, M. C., Griswold, J. A., Auer, M., Hamood, A. N., and Rumbaugh, K. P. (2007). Pseudomonas aeruginosa forms biofilms in acute infection independent of cell-to-cell signaling. Infect. Immun. 75, 3715-3721.
Schiessl, K. T., Hu, F., Jo, J., Nazia, S. Z., Wang, B., Price-Whelan, A., Min, W., and Dietrich, L. E. P. (2019). Phenazine production promotes antibiotic tolerance and metabolic heterogeneity in Pseudomonas aeruginosa biofilms. Nat. Commun. 10, 762.
Schneider, C. A., Rasband, W. S., and Eliceiri, K. W. (2012). NIH Image to ImageJ: 25 years of image analysis. Nat. Methods 9, 671-675.
Schreiber, F., and Ackermann, M. (2019). Environmental drivers of metabolic heterogeneity in clonal microbial populations. Curr. Opin. Biotechnol. 62, 202-211.
Schreiber, F., Littmann, S., Lavik, G., Escrig, S., Meibom, A., Kuypers, M. M. M., and Ackermann, M. (2016). Phenotypic heterogeneity driven by nutrient limitation promotes growth in fluctuating environments. Nat. Microbiol. 1, 16055.
Schreiber, K., Boes, N., Eschbach, M., Jaensch, L., Wehland, J., Bjarnsholt, T., Givskov, M., Hentzer, M., and Schobert, M. (2006). Anaerobic survival of Pseudomonas aeruginosa by pyruvate fermentation requires an Usp-type stress protein. J. Bacteriol. 188, 659-668.
Shah, S., Lubeck, E., Zhou, W., and Cai, L. (2016). In situ transcription profiling of single cells reveals spatial organization of cells in the mouse hippocampus. Neuron 92, 342-357.
Shah, S., Takei, Y., Zhou, W., Lubeck, E., Yun, J., Eng, C.-H. L., Koulena, N., Cronin, C., Karp, C., Liaw, E. J., et al. (2018). Dynamics and spatial genomics of the nascent transcriptome by intron seqFISH. Cell 174, 363-376.e16.
Shi, H., Shi, Q., Grodner, B., Lenz, J. S., Zipfel, W. R., Brito, I. L., and De Vlaminck, I. (2020). Highly multiplexed spatial mapping of microbial communities. Nature 588, 676-681.
Skinner, S. O., Sephlveda, L. A., Xu, H., and Golding, I. (2013). Measuring mRNA copy number in individual Escherichia coli cells using single-molecule fluorescent in situ hybridization. Nat. Protoc. 8, 1100-1113.
So, L.-H., Ghosh, A., Zong, C., Sephlveda, L. A., Segev, R., and Golding, I. (2011). General properties of transcriptional time series in Escherichia coli. Nat. Genet. 43, 554-560.
Stewart, P. S. (2003). Diffusion in biofilms. J. Bacteriol. 185, 1485-1491.
Stewart, P. S., and Franklin, M. J. (2008). Physiological heterogeneity in biofilms. Nat. Rev. Microbiol. 6, 199-210.
Stylianidou, S., Brennan, C., Nissen, S. B., Kuwada, N.J., and Wiggins, P. A. (2016). SuperSegger: robust image segmentation, analysis and lineage tracking of bacterial cells. Mol. Microbiol. 102, 690-700.
Su, S., Panmanee, W., Wilson, J. J., Mahtani, H. K., Li, Q., Vanderwielen, B. D., Makris, T. M., Rogers, M., McDaniel, C., Lipscomb, J. D., et al. (2014). Catalase (KatA) plays a role in protection against anaerobic nitric oxide in Pseudomonas aeruginosa. PLoS One 9, e91813.
Takei, Y., Yun, J., Zheng, S., Ollikainen, N., Pierson, N., White, J., Shah, S., Thomassie, J., Suo, S., Eng, C.-H. L., et al. (2021). Integrated spatial genomics reveals global architecture of single nuclei. Nature 590, 344-350.
Toyofuku, M., Nomura, N., Fujii, T., Takaya, N., Maseda, H., Sawada, I., Nakajima, T., and Uchiyama, H. (2007). Quorum sensing regulates denitrification in Pseudomonas aeruginosa PAO1. J. Bacteriol. 189, 4969-4972.
Tropini, C., Earle, K. A., Huang, K. C., and Sonnenburg, J. L. (2017). The gut microbiome: connecting spatial organization to function. Cell Host Microbe 21, 433-442.
Turnbull, L., Toyofuku, M., Hynen, A. L., Kurosawa, M., Pessi, G., Petty, N. K., Osvath, S. R., CarcamoOyarce, G., Gloag, E. S., Shimoni, R., et al. (2016). Explosive cell lysis as a mechanism for the biogenesis of bacterial membrane vesicles and biofilms. Nat. Commun. 7, 11220.
Vacheron, J., Heiman, C. M., and Keel, C. (2021). Live cell dynamics of production, explosive release and killing activity of phage tail-like weapons for Pseudomonas kin exclusion. Commun. Biol. 4, 87.
Vallet-Gely, I., and Boccard, F. (2013). Chromosomal organization and segregation in Pseudomonas aeruginosa. PLoS Genet. 9, e1003492.
Veening, J.-W., Smits, W. K., and Kuipers, O. P. (2008). Bistability, epigenetics, and bet-hedging in bacteria. Annu. Rev. Microbiol. 62, 193-210.
Wessel, A. K., Arshad, T. A., Fitzpatrick, M., Connell, J. L., Bonnecaze, R. T., Shear, J. B., and Whiteley, M. (2014). Oxygen limitation within a bacterial aggregate. mBio 5:e00992.
Whitchurch, C. B., Tolker-Nielsen, T., Ragas, P. C., and Mattick, J. S. (2002). Extracellular DNA required for bacterial biofilm formation. Science 295, 1487.
Wilbert, S. A., Mark Welch, J. L., and Borisy, G. G. (2020). Spatial ecology of the human tongue dorsum microbiome. Cell Rep. 30, 4003-4015.e3.
Wolf, F. A., Angerer, P., and Theis, F. J. (2018). SCANPY: large-scale single-cell gene expression data analysis. Genome Biol. 19, 15.
Wolfsberg, E., Long, C. P., and Antoniewicz, M. R. (2018). Metabolism in dense microbial colonies: 13C metabolic flux analysis of E. coli grown on agar identifies two distinct cell populations with acetate crossfeeding.
Metab. Eng. 49, 242-247.
Wurtzel, O., Yoder-Himes, D. R., Han, K., Dandekar, A. A., Edelheit, S., Greenberg, E. P., Sorek, R., and
Lory, S. (2012). The single-nucleotide resolution transcriptome of Pseudomonas aeruginosa grown in body temperature. PLoS Pathog. 8, e1002945.
Yoon, S. S., Hennigan, R. F., Hilliard, G. M., Ochsner, U. A., Parvatiyar, K., Kamani, M. C., Allen, H. L., DeKievit, T. R., Gardner, P. R., Schwab, U., et al. (2002). Pseudomonas aeruginosa anaerobic respiration in biofilms. Dev. Cell 3, 593-603.
Zaborina, O., Holbrook, C., Chen, Y., Long, J., Zaborin, A., Morozova, I., Fernandez, H., Wang, Y., Turner, J. R., and Alverdy, J. C. (2008). Structure-function aspects of PstS in multi-drug-resistant Pseudomonas aeruginosa. PLoS Pathog. 4, e43.

What we claim:

1. A method, comprising steps of:
(a) providing two or more samples of cells;
(b) labelling the cells of each sample with one or more sample probes,
wherein the sample probes interact with one or more sample identifiers;
(c) treating each sample in the two or more samples to different conditions;
(d) combining the two or more samples to create a pooled sample;
(e) barcoding one or more targets in the pooled sample;
(f) imaging the barcodes; and
(g) demultiplexing the sample probes to associate cells with their samples.

2. The method of claim 1, wherein step (b) is after step (c).

3. The method of claim 1, wherein step (e) is before step (d).

4. The method of claim 1, wherein step (g) is before step (f).

5. The method of claim 1, wherein step (g) is before step (e).

6. The method of claim 1, wherein:
step (b) is after step (c);
step (e) is before step (d);
step (g) is before step (f); and
step (g) is before step (e).

7. The method of any one of claims 1-6, wherein the barcoding comprises:
contacting each sample in the one or more samples with a first plurality of target probes, so that the target probes interact with one or more targets.

8. The method of claim 7, wherein the target probes are contacted to the two or more samples before the two or more samples are pooled.

9. The method of any one of claims 1-8, wherein the imaging comprises:
imaging the pooled sample after the first contacting step so that interaction of the target probes with their targets is detected.

10. The method of claim 9, wherein the method further comprises:
(h) repeating the contacting and imaging steps, each time with a new plurality of target probes so that a target in the sample is described by a barcode, and can be differentiated from another target in the sample by a difference in their barcodes.

11. The method of any one of claims 1-10, wherein the samples comprise bacterial cells, archaeal cells, eukaryotic cells, or a combination thereof.

12. The method of any one of claims 1-10, wherein the samples are tissues, cells, or extracts from cells.

13. The method of any one of claims 1-10, wherein the samples are biofilms.

14. The method of any one of claims 1-10, wherein the samples are from patients.

15. The method of any one of claims 1-10, wherein the different conditions the samples are treated comprise different growth conditions, different chemical exposures, different environmental conditions, or combinations thereof.

16. The method of any one of claims 1-10, wherein the samples comprise exogeneous sample identifiers, endogenous sample identifiers, or any combination thereof that can be barcoded for identification.

17. The method of any one of claims 1-10, wherein the samples comprise specific genes that can be turned “ON” or turned “OFF” in the presence of a signal.

18. The method of any one of claims 1-10, wherein 3, 4, 5, 6, 7, 8, 9, 10, 11, 12, 13, 14, 15, 20, or 25 different samples are pooled.

19. The methods of any one of claims 1-10, wherein the sample identifiers are selected from transcripts, RNA, DNA loci, chromosomes, DNA, proteins, lipids, glycans, cellular targets, organelles, and any combinations thereof.

20. The method of any one of claims 1-10, wherein the sample identifiers are selected from synthetic RNA, ribosomal RNA, 16S RNA, 18S RNA, and lncRNA.

21. The method of any one of claims 1-10, wherein the sample identifiers differentiate one organism from another.

22. The method of any one of claims 1-10, wherein each sample probe is selected from proteins, modified proteins, RNA, oligonucleotides, antibodies, antibody fragments, and combinations thereof.

23. The method of claim 22, wherein each sample probe comprises an oligonucleotide.

24. The method of claim 23, wherein the oligonucleotides interact with identifier regions on sample identifiers by hybridization.

25. The method of claim 23, where the oligonucleotide has a sequence that is complementary to the sample identifier region, and wherein the sequence complementarity is at least 60%, 65%, 70%, 75%, 80%, 85%, 90%, 91%, 92%, 93%, 94%, 95%, 96%, 97%, 98%, 99%, or 100%.

26. The method of claim 23, wherein the oligonucleotide is at least 6 nucleotides long.

27. The method of claim 23, wherein the oligonucleotide is at least 17 nucleotides long.

28. The method of claim 23, wherein the oligonucleotide is at least 25 nucleotides long.

29. The method of claim 23, wherein the oligonucleotide is at least 30 nucleotides long.

30. The method of claim 23, wherein the sample probe comprises a detectable label.

31. The method of anyone of claims 1-10, wherein the sample identifier comprises a sample readout probe binding site or an intermediate probe binding site.

32. The method of any one of claims 22-31, wherein the sample identifier interacts with one or more sample intermediate probes.

33. The method of claim 32, wherein the sample intermediate probe is selected from proteins, modified proteins, RNA, oligonucleotides, antibodies, antibody fragments, and combinations thereof.

34. The method claim 33, wherein the sample intermediate probe comprises an oligonucleotide.

35. The method of claim 34, wherein the sample intermediate probe comprises a sequence complementary to the sample identifier and a sequence complementary to a sample readout probe.

36. The method of claim 34, wherein the sample intermediate probe comprises a sequence complementary to a sample identifier and an overhang sequence.

37. The method of claim 36, wherein the overhang sequence is complementary to a sample readout probe.

38. The method of claim 37, wherein the overhang sequence is complementary to a sample bridge probe.

39. The method of claim 38, wherein the sample bridge probe is complementary to a sample readout probe and to a sample intermediate probe.

40. The method of claim 33, wherein the sample intermediate probes are preserved through multiple contacting and imaging steps.

41. The method of claim 35, wherein the sample readout probe binding site is at least 10 nucleotides long.

42. The method of claim 41, wherein the sample readout probe is an oligonucleotide comprising a detectably moiety.

43. The method of claim 42, wherein the sample readout probe has a sequence complementary to the readout probe binding site, wherein the sequence complementarity is at least 60%, 65%, 70%, 75%, 80%, 85%, 90%, 91%, 92%, 93%, 94%, 95%, 96%, 97%, 98%, 99%, or 100%.

44. The method of any one of claims 1-10, wherein the targets are selected from proteins, modified proteins, transcripts, RNA, DNA loci, exogenous proteins, exogenous nucleic acids, hormones, carbohydrates, small molecules, biologically active molecules, and combinations thereof.

45. The method of any one of claims 1-10, wherein the targets are selected from nucleic acids or proteins involved in biosynthetic capacity, anaerobic physiology, stress responses, cellular signaling, biofilm matrix components, motility, all major quorum-sensing (QS) systems, multiple antibiotic resistance and core virulence factors.

46. The method of any one of claims 7-10, wherein the target probes are selected from proteins, modified proteins, RNA, oligonucleotides, antibodies, antibody fragments, and combinations thereof.

47. The method of any one of claims 7-10, wherein the target probes interact with their targets through one or more target intermediate probes.

48. The method of claim 47, wherein the target intermediate probes hybridize to targets.

49. The method of claim 47, wherein each target intermediate probe comprises a sequence complementary to its target and an overhang sequence.

50. The method of claim 48, wherein the overhang sequence is complementary to a target readout probe.

51. The method of claim 50, wherein the overhang sequence is complementary to a target bridge probe.

52. The method of claim 51, wherein each target bridge probe is complementary to a target readout probe and to an intermediate probe.

53. The method of claim 47, wherein the target intermediate probes are preserved through multiple contacting and imaging steps.

54. The method of claim 50, wherein each target readout probe comprises a detectable label.

55. The method of any of claims 7-10 and 46, wherein each target probe is detectably labelled.

56. The method of any one of claims 7-10, wherein at least one contacting step differs from another contacting step in the labelling of at least one of the targets.

57. The method of any one of claims 7-10, wherein each target probe in the first plurality of probes is labelled with a detectably moiety.

58. The method of any one of claims 7-10, wherein each target probe comprises a detectable moiety and at least one contacting step differs from another contacting step by having a different detectable moiety for each target.

59. The method of any one of claims 7-10, wherein at least two different target probes interact with a first target and wherein at least two different target probes interact with a second target.

60. The method of any one of claims 7-10, wherein the target probes comprise one or more labels selected from two, three, or four different labels.

61. The method of any one of claims 7-10, wherein the barcode for the target in the sample includes a signal that is amplified.

62. The method of any one of claims 7-10, wherein each target is different.

63. The method of any one of claims 7-10, wherein the target probes each comprise the same detectable moiety and the same sequence.

64. The method of any one of claims 7-10, wherein each target probe interacts with its target through one or more intermediate probes each of which is hybridized to the target.

65. The method of any one of claims 1-10, further comprising analyzing cell size and shape, markers, immunofluorescence measurements, or any combinations thereof.

66. The method of claim 1, wherein demultiplexing comprises:
imaging the pooled sample so that interaction of the sample probes with their sample identifiers is detected.

67. The method of claim 66, wherein demultiplexing comprises:
analyzing background and signals generated by the sample readout probes interacting with the sample probes, the sample probes interacting with the sample identifiers within segmented boundaries to provide a signal-to-background score for each readout; and
classifying the cells according to the positive readout signals.

68. The method of any one of claims 1-10 and 66-67, further comprising removing the readout probes after one or more imaging steps.

69. The method of claim 68, wherein the step of removing comprises contacting the plurality of readout probes with an enzyme that digests a readout probe.

70. The method of claim 68, wherein the step of removing comprises contacting the plurality of target readout probes with a DNase, contacting the plurality of target probes with an RNase, photobleaching, strand displacement, formamide wash, heat denaturation, or combinations thereof.

71. The method of claim 70, wherein the target readout probes are removed by photobleaching.

72. The method of any one of claims 1-10, further comprising clearing the sample.

73. The method of claim 72, wherein the sample is cleared by CLARITY.

74. The method of claim 72, wherein the sample is cleared following hydrogel embedding.
